(19) United States
(12) Patent Application Publication
(10) Pub. No.: US 2011/0039258 A1
(43) Pub. Date: Feb. 17, 2011

(54) METHODS AND COMPOSITIONS FOR DIFFERENTIAL EXPANSION OF FETAL CELLS IN MATERNAL BLOOD AND THEIR USE

(75) Inventors: Patricia McNeeley, San Diego, CA (US); Philippe Marchand, Poway, CA (US); Jonathan Diver, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: CELULA INC., San DIego, CA (US)

(21) Appl. No.: 12/445,732

(22) PCT Filed: Oct. 15, 2007
(86) PCT No.: PCT/US07/81396

§ 371 Date: Nov. 1, 2010

Related U.S. Application Data
(60) Provisional application No. 60/829,668, filed on Oct. 16, 2006.

(51) Int. Cl. C12Q 1/68; C12N 5/02; C12Q 1/02

(52) U.S. Cl. 435/6; 435/373; 435/173.9; 435/29

(57) ABSTRACT

Disclosed is a method and compositions for the differential expansion of fetal cells over maternal cells. In the method, cells from a sample of maternal blood containing CD34+ cells of both maternal and fetal origin are incubated in the presence of Stem Cell Factor in serum free media. It has been discovered that incubation of fetal cells in the presence of SCF will preferentially expand the fetal cells relative to adult cells. Fetal cells can also be identified, enriched or obtained by differential expansion of the fetal cells during colony formation. It has been discovered that differential expansion of fetal cells can result in colonies of fetal cells that are larger than colonies of adult cells. The fetal CD34+ cells can be expanded without generation of significant clonal genetic artifacts during expansion. Also disclosed is a method and compositions for producing differentiated fetal cells. It has been discovered that differentiated fetal cells have markers that distinguish the fetal cells from adult cells. Also disclosed are fetal cells made or obtained using the disclosed methods. For example, disclosed are expanded and/or differentiated fetal cells. The disclosed fetal cells can be used for any purpose and in any way that fetal cells can be used. The disclosed fetal cells are particularly useful for prenatal analysis of a gestating fetus.

### CROSS-REFERENCE TO RELATED APPLICATIONS

This application claims benefit of U.S. Provisional Application No. 60/829,668, filed Oct. 16, 2006. U.S. Application No. 60/829,668, filed Oct. 16, 2006, is hereby incorporated herein by reference in its entirety.

### FIELD OF THE INVENTION

The disclosed invention is generally in the field of fetal cells and specifically in the area of fetal cell analysis.

### BACKGROUND OF THE INVENTION

Prenatal diagnostic methods are primarily aimed at obtaining genetic information on a fetus or an embryo. The search for genetic information on a fetus generally involves identifying the presence of a specific allele of a given gene or a combination of alleles on a given fetal DNA sequence, genetically associating a fetal DNA polymorphism with a particular allele, or detecting chromosomal abnormalities. One major application of prenatal genetic diagnosis concerns the detection of congenital anomalies.

Prenatal genetic diagnostic methods used in clinical practice essentially involve invasive techniques such as amniocentesis, the removal of chorionic villi, the removal of fetal blood or tissue biopsies. Those techniques involve obtaining samples directly from the fetus or indirectly from ovular structures. Because of the highly invasive nature of those methods, they are prone to complications for the mother or the fetus. Examples of such complications which can be cited in the case of amniocentesis are the risk of infection, feto-maternal hemorrhage with possible allo-immunization, loss of amniotic fluid and abdominal pain. Different studies have estimated the risk of a miscarriage after amniocentesis at 0.2% to 2.1% higher than that of the control group. As a result, amniocentesis is only suggested for women in whom the risk of having a child with a genetic abnormality exceeds that of iatrogenic miscarriage.

In order to limit the use of invasive prenatal diagnostic techniques risking the complications mentioned above and which are generally disagreeable and/or the source of stress for the mother, the development of non-invasive methods constitutes a major aim in modern obstetrics.

In particular, fetal cells circulating in maternal blood constitutes a source of genetic material that is of potential use for prenatal genetic diagnosis (Bianchi, Br J Haematol 1999 105: 574-583; Fisk, Curr Opin Obstet Gynecol 1998 10: 81-83). During pregnancy, different cell types of fetal origin traverse the placenta and circulate in the maternal blood (Bianchi, Br J Haematol 1999 105: 574-583). Such cell types include lymphoid and erythroid cells, myeloid precursors and trophoblastic epithelial cells (cytotrophoblasts and syncytiotrophoblasts).

Methods for analyzing the genome of fetal cells circulating in maternal blood with a view to prenatal diagnosis have been described, but they remain relatively limited as regards sensitivity and the specificity of the diagnosis (Di Naro et al., Mol Hum Reprod 2000 6: 571-574; Watanabe et al., Hum Genet 1998 102: 611-615; Takabayashi et al., Prenat Diagn 1995 15: 74-77; Sekizawa et al., Hum Genet 1998 102: 393-396). The advantage in developing a non-invasive, highly specific prenatal diagnosis method results from the possibility of using it to reduce the proportion of invasive diagnostic methods carried out in pregnant women for whom the result is negative in the end. By way of example, in the case of trisomy 21, which concerns one woman in 700, prenatal diagnosis is currently offered in France only if the mother is 38 years old, while a biochemical analytical test capable of detecting 60% of trisomy 21s for 5% of the price of amniocentesis is proposed for younger women. However, 40% of trisomy 21 cases are not detected by currently available tests. Prenatal detection of trisomy 21 in fetal cells isolated from the maternal plasma using a FISH technique has been described. That approach is interesting, but as fetal cells are rare in plasma (1 in 500 to 1 in 2000) and often include apoptotic cells, reliable diagnosis would require carrying out the method on a very large number of cells, rendering it impossible to carry out routinely. Further, euploid fetal cells cannot be identified by that approach.

One limitation of such approaches derives from the fact that fetal cells circulating in the blood are present in very low concentrations. Studies based on PCR detection of the Y chromosome in blood samples without prior selection have allowed the mean number of fetal cells to be determined to be about 1 fetal lymphocyte cell per milliliter of blood (Bianchi, J Perinat Med 1998 26: 175-85). Further, it has been shown that fetal cells of myeloid or lymphoid origin (CD34 or CD38 positive) are still present in maternal blood up to 27 years after pregnancy or miscarriage (Bianchi et al., PNAS 1996, 93: 705). When isolating them, then, it is not certain that they derive from the current pregnancy. Thus, there is a need for improved prenatal diagnosis of maternal blood.

### BRIEF SUMMARY OF THE INVENTION

Disclosed is a method and compositions for the differential expansion of fetal cells over maternal cells. In the method, cells from a sample of maternal blood containing CD34+ cells of both maternal and fetal origin are incubated in the presence of Stem Cell Factor (SCF) in serum free media. It has been discovered that incubation of fetal cells in the presence of SCF will preferentially expand the fetal cells relative to adult cells. Such expansion can be combined with other preparation, isolation, sorting, selection and enrichment of fetal cells and/or CD34+ cells both as described herein and as known in the art. Also disclosed is method and compositions for expansion of fetal cells, where CD34+-enriched cells from maternal blood are incubated in the presence of SCF and serum free medium such as, for example, Hematopoietic Progenitor Growth Medium (HPGM).

Differential expansion of fetal cells (also referred to herein as preferential expansion) can be any increase in the number or proportion of fetal dells relative to adult cells. For example, fetal CD34+ cells can be expanded to a ratio of at least about 5 with adult CD34+ cells. Fetal CD34+ cells can be preferentially expanded by at least about 5 fold relative to adult CD34+ cells. Fetal CD34+ cells can be differentially expanded by a factor of at least about 5 compared with adult CD34+ cells. Fetal cells can be differentially expanded by a factor of at least about 5 compared with adult cells. Fetal CD34+ cells can be expanded to a ratio of at least about 3 with adult CD34+ cells. Fetal CD34+ cells can be preferentially expanded by at least about 3 fold relative to adult CD34+ cells. Fetal CD34+ cells can be differentially expanded by a factor of at least about 3 compared with adult CD34+ cells. Fetal cells can be differentially expanded by a factor of at least about 3 compared with adult cells. The fetal CD34+ cells can be preferentially expanded by at least about 20 fold relative to adult CD34+ cells.

Also disclosed is a method and compositions for producing differentiated fetal cells. In the method, CD34+-enriched cells from maternal blood are incubated under conditions that promote differentiation of fetal CD34+ cells into or on one or more predetermined developmental pathways. It has been discovered that differentiated fetal cells have markers that distinguish the fetal cells from adult cells. Differentiated fetal CD34+ cells can be identified based on one or more cell markers, such as cell surface markers. The conditions that promote differentiation of fetal CD34+ cells can include the presence of Stem Cell Factor. The cell marker can be CD1c, CD14, CD24, CD48, CD86, CD235a, MPO, MS4A6A, MS4A7, and ASGR2, or a combination.

The differentially expanded cells can be CD34+ cells. The fetal cells can be differentially expanded by a factor of at least about 2, 3, 4, 5, 6, 7, 8, 9, 10, 11, 12, 13, 14, 15, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, or 200 compared with maternal cells. The fetal cells can be differentially expanded to a ratio of at least about 2, 3, 4, 5, 6, 7, 8, 9, 10, 11, 12, 13, 14, 15, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, or 200 compared with maternal cells. The fetal cells can be preferentially expanded by at least about 2, 3, 4, 5, 6, 7, 8, 9, 10, 11, 12, 13, 14, 15, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, or 200 fold compared with maternal cells. The fetal cells can be differentially expanded by a factor of at least about 2, 3, 4, 5, 6, 7, 8, 9, 10, 11, 12, 13, 14, 15, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, or 200 compared with adult cells or adult CD34+ cells.

The cells from maternal blood can be incubated in the presence of SCF at a concentration of from about 15, 20, 25, 30, 35, 40, 45, or 50 ng/ml to about 12.5, 25, 50, 55, 60, 65, 70, 75, 80, 85, 90, 95, 100, 105, 110, 115, 120, 125, 130, 135, 140, 145, 150, 200, or 250 ng/ml. The cells from maternal blood can be incubated in the presence of SCF at a concentration of about 12.5, 15, 20, 25, 30, 35, 40, 45, 50, 55, 60, 65, 70, 75, 80, 85, 90, 95, 100, 105, 110, 115, 120, 125, 130, 135, 140, 145, or 150 ng/ml.

The cells from maternal blood can be incubated in the presence of Interleukin-6 (IL-6). The cells from maternal blood can be incubated in the presence of IL-6 at a concentration of from about 1, 2, 3, 4, 5, 6, 7, 8, 9, or 10 ng/ml to about 11, 12, 13, 14, 15, 16, 17, 18, 19, 20, 21, 22, 23, 24, or 25 ng/ml. The cells from maternal blood can be incubated in the presence of IL-6 at a concentration of about 1, 2, 3, 4, 5, 6, 7, 8, 9, 10, 11, 12, 13, 14, 15, 16, 17, 18, 19, 20, 21, 22, 23, 24, or 25 ng/ml.

The cells from maternal blood can be incubated in the presence of Interleukin-3 (IL-3). The cells from maternal blood can be incubated in the presence of IL-3 at a concentration of from about 1, 2, 3, 4, 5, 6, 7, 8, 9, 10, 11, 12, 13, 14, or 15 ng/ml to about 16, 17, 18, 19, 20, 21, 22, 23, 24, 25, 26, 27, 28, 29, or 30 ng/ml. The cells from maternal blood can be incubated in the presence of IL-3 at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml.

The cells from maternal blood can be incubated in the presence of SCF at a concentration of from about 50 ng/ml, IL-6 at a concentration of about 5 ng/ml, and IL-3 at a concentration of about 10 ng/ml. The cells from maternal blood can be incubated in the presence of one or more of IL-3, IL-6, erythropoietin (EPO), thrombopoietin (TPO), Flt-1, Flt-3, IL-1, IL-11, GM-CSF, G-CSF, Wnt, Notch, IGF, Bone Morphogenic Protein (BMP), Sonic Hedgehog, CxCL12, basic fibroblast growth factor or specific vitamins or specific antibodies capable of inhibiting adult cell growth.

The cells from maternal blood can be incubated in the presence of IL-3 and/or IL-6. The cells from maternal blood can be incubated in the absence of IL-3, IL-6, TPO and/or EPO. The cells from maternal blood can be incubated in the presence of SCF at a concentration of from about 100 ng/ml. The cells from maternal blood can be incubated in the absence of or without supplementation with Flt-3 ligand and TPO, in the absence of or without supplementation with IL-3 and IL-6, in the absence of or without supplementation with TPO and EPO, in the absence of or without supplementation with EPO, in the absence of or without supplementation with serum, in the absence of or without supplementation with cytokines other than SCF, or a combination.

The fetal CD34+ cells can be expanded in the absence of significant or substantial expansion of adult cells. The fetal CD34+ cells can be expanded without generation of significant clonal genetic artifacts during expansion. The clonal genetic artifacts can be can be clinically significant genetic artifacts. Clinically significant genetic artifacts are genetic changes induced by growth of cells that can be detected in a genetic assay to which the cells are subjected.

Fetal cells and CD34+ cells can be enriched from maternal blood. For example, fetal cells can be enriched from maternal blood by selecting or sorting cells based on the presence or absence of the markers CD34, CD133, CD117, CD2, CD45, HLA, Lineage, and/or CD90, or by removing or lysing red blood cells, by selecting or sorting cells based on the presence or absence of one or more of the markers, or a combination. Fetal cells can be enriched from maternal blood by immunomagnetic selection. CD34+ cells can be enriched from maternal blood by positive selection of CD34+ cells, by direct selection of CD34+ cells, by indirect selection of CD34+ cells, by depletion of non-CD34+ cells, by depletion of CD34− cells, or by a combination. CD34+ cells can be enriched from maternal blood by selecting or sorting cells based on the presence or absence of one or more fetal cell markers. The fetal cell markers can be CD1c, CD14, CD24, CD48, CD86, CD235a, MPO, MS4A6A, MS4A7, and ASGR2 or a combination of these markers. CD34+ cells can be enriched from maternal blood by positive selection of CD34+ cells and by depletion of CD38+ cells and GlycophorinA+ cells. This generally can be done prior to expansion of the cells.

The fetal CD34+ cells can form colonies. The fetal CD34+ cells can form clonal colonies larger than colonies formed by the adult CD34+ cells. This can allow identification of fetal cells from maternal blood. One or more colonies of fetal CD34+ cells can be harvested. The fetal cells from maternal blood can be incubated in the presence of one or more support cells.

Differentially expanded and/or enriched fetal and/or CD34+ cells can be differentiated into one or more predetermined developmental pathways, whereby the differentiated fetal CD34+ cells differ from the differentiated adult CD34+ cells in one or more cell markers. Differentiated fetal CD34+ cells can be distinguished from differentiated adult CD34+ cells by assessing one or more cell markers. The differentiated fetal CD34+ cells can differ from differentiated adult CD34+ cells in one or more cell markers. The differentiated fetal CD34+ cells can be identified by distinguishing differentiated fetal CD34+ cells from differentiated adult CD34+ cells by assessing one or more cell markers. The differentiated fetal CD34+ cells can form colonies. The differentiated fetal CD34+ cells can form colonies larger than colonies formed by the adult CD34+ cells. One or more colonies of fetal CD34+ cells can be harvested.

The CD34+ cells can be differentiated prior to, simultaneous with, or following expansion of the fetal CD34+ cells. The expanded fetal CD34+ cells can be differentiated. The fetal CD34+ cells can be differentiated during expansion of the fetal CD34+ cells.

Differentially expanded and/or enriched fetal and/or fetal CD34+ cells can be selecting or sorting from adult cells based on one or more cell markers. The marker can be CD1c, CD14, CD24, CD48, CD86, CD235a, MPO, MS4A6A, MS4A7, and ASGR2, or a combination.

Also disclosed is a method of analyzing one or more of the fetal cells for one or more characteristics. The fetal cells can be fetal cells obtained, expanded and/or differentiated as described herein. The fetal cells can form colonies and one or more colonies of fetal cells can be harvested, wherein one or more of the expanded fetal CD34+ cells that are analyzed are derived from one or more of the harvested colonies.

The characteristic can be genotype, phenotype, physiological function, biochemical function, or a combination. The characteristic can be the presence or absence of one or more particular nucleic acid sequences. The characteristic can be the sex of the fetus from which the fetal cells derived. The sex of the fetus can be analyzed by detecting the presence of Y chromosomes, X chromosomes, or both in the fetal cells.

The characteristic can be a disease or condition or an indicator of a disease or condition. The indicator of the disease or condition can be analyzed by detecting one or more mutations, single nucleotide polymorphisms, genetic markers, or a combination associated with the disease or condition. The mutation, single nucleotide polymorphism, or genetic marker can be, for example, a cystic fibrosis-associated mutation, single nucleotide polymorphism, or genetic marker, a Duchenne muscular dystrophy-associated mutation, single nucleotide polymorphism, or genetic marker, a hemophilia A-associated mutation, single nucleotide polymorphism, or genetic marker, a Gaucher disease-associated mutation, single nucleotide polymorphism, or genetic marker, a sickle cell anemia-associated mutation, single nucleotide polymorphism, or genetic marker, a Tay-Sachs-associated mutation, single nucleotide polymorphism, or genetic marker, or a combination

The characteristic can be a chromosomal abnormality. The chromosomal abnormality can be chromosomal aneuploidy, chromosomal translocation, deletion, duplication or a combination. The chromosomal aneuploidy can be trisomy 21, trisomy 18, trisomy 13 or a combination.

Also disclosed are fetal cells made or obtained using the disclosed methods. For example, disclosed are fetal cells obtained by incubating CD34+-enriched cells from maternal blood in the presence of Stem Cell Factor and Hematopoietic Progenitor Growth Medium, whereby fetal CD34+ cells are differentially expanded by a factor of at least about 5 compared with adult CD34+ cells. Also disclosed are differentiated fetal cells obtained by incubating CD34+-enriched cells from maternal blood under conditions that promote differentiation of fetal CD34+ cells into one or more predetermined developmental pathways, wherein conditions that promote differentiation of fetal CD34+ cells include the presence of Stem Cell Factor and identifying differentiated fetal CD34+ cells based on one or more cell markers, wherein the cell surface marker is CD1c, CD14, CD24, CD48, CD86, CD235a, MPO, MS4A6A, MS4A7, and ASGR2, or a combination.

Also disclosed are compositions that includes a mixture of fetal and maternal stem cells wherein the fetal cells are present at a concentration of greater than 5 times that of the maternal cells: Also disclosed are compositions that includes a mixture of fetal and maternal stem cells wherein the fetal cells are present at a concentration of greater than 3 times that of the maternal cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION OF THE INVENTION

The disclosed method and compositions may be understood more readily by reference to the following detailed description of particular embodiments and the Example included therein and to the Figures and their previous and following description.

Disclosed is a method and compositions for the differential expansion of fetal cells over maternal cells. In the method, cells from a sample of maternal blood containing CD34+ cells of both maternal and fetal origin are incubated in the presence of Stem Cell Factor in serum free media. It has been discovered that incubation of fetal cells in the presence of SCF will preferentially expand relative to adult cells despite the phenotypic similarity of the fetal and maternal cells prior to expansion. Other factors can be used in the method, such as Hematopoietic Progenitor Growth Medium, IL-6, IL-3, EPO, TPO, Flt-1, Flt-3, IL-1, IL-11, GM-CSF, G-CSF, Wnt, Notch, IGF, BMP, Sonic Hedgehog, CxCL12, basic fibroblast growth factor or specific vitamins or specific antibodies capable of inhibiting adult cell growth. Such other factors can also be absent. For example, cells from maternal blood can be incubated in the absence of IL-3, IL-6, TPO and/or EPO.

As used herein, “fetal cell” refers to cells of or that are derived from an embryo or fetus. Cells of or derived from an embryo or fetus can be referred to as being of fetal origin. As used herein, “maternal cell” refers to cells that are cells of or derived from a pregnant subject. The term maternal cell excludes cells of or derived from a genetically distinct subject, and in particular excludes cells of any embryo or fetus of the pregnant subject. Cells of or derived from a pregnant subject can be referred to as being of maternal origin. Maternal cells can also be referred to herein as adult cells. Fetal cells are not adult cells. “Maternal blood” refers to blood of or derived from a pregnant subject. As used herein, “subject” refers to an animal, human or non-human. Pregnant subjects are mammalian subjects. As used herein, “incubation” refers to exposing and/or maintaining stated components under stated conditions.

As used herein, “differential expansion” and “preferential expansion” refer to an expansion or increase in one or more compositions, cells, or characteristics (or the level or quantity thereof) relative to one or more other compositions, cells, or characteristics (or the level or quantity thereof). Differential expansion can result in a change in proportion or ratio between the compositions, cells, or characteristics (or the level or quantity thereof) subject to differential expansion. For example, differential expansion of fetal cells can be any increase in the number or proportion of fetal cells relative to adult cells. For example, fetal CD34+ cells can be expanded to a ratio of at least about 5 with adult CD34+ cells. Fetal CD34+ cells can be preferentially expanded by at least about 5 fold relative to adult CD34+ cells. Fetal CD34+ cells can be differentially expanded by a factor of at least about 5 compared with adult CD34+ cells. Fetal cells can be differentially expanded by a factor of at least about 5 compared with adult cells. Fetal CD34+ cells can be expanded to a ratio of at least about 3 with adult CD34+ cells. Fetal CD34+ cells can be preferentially expanded by at least about 3 fold relative to adult CD34+ cells. Fetal CD34+ cells can be differentially expanded by a factor of at least about 3 compared with adult CD34+ cells. Fetal cells can be differentially expanded by a factor of at least about 3 compared with adult cells. The fetal CD34+ cells can be preferentially expanded by at least about 20 fold relative to adult CD34+ cells.

Fetal cells can also be identified, enriched or obtained by differential expansion of the fetal cells during colony formation. It has been discovered that differential expansion of fetal cells can result in colonies of fetal cells that are larger than colonies of adult cells. For example, plating and incubation of cells from maternal blood in the presence of SCF will produce colonies of fetal cells that are larger than colonies of adult cells. The fetal cells can be harvested. As used herein, “harvested” refers to removal from a growth or storage location or condition. Cells can be confirmed as fetal cells by identification of fetal cell-specific features, such as fetal cell markers. For example, cells can be labeled via fetal cell markers. Any detection technique can be used, including destructive techniques since only a portion of a colony need be assayed. As another example, harvested cells can be sorted based on fetal cell markers. As another example, colonies can be labeled in situ.

The fetal cells can be expanded in the absence of significant or substantial expansion of adult cells. The fetal cells can be expanded without generation of significant clonal genetic artifacts during expansion. Clonal genetic artifacts can be clinically significant genetic artifacts. Clinically significant genetic artifacts are genetic changes induced by growth of cells that can be detected in a genetic assay to which the cells are subjected. Thus, for example, a lack of detectable changes in one or more cell markers can indicate that no significant clonal genetic artifacts were generated during expansion. Whether a feature of a cell is or is not a clonal genetic artifact can be defined in terms of the cells and the genetic feature(s) that are assayed. Thus, for example, an expanded fetal cell may have a genetic abnormality but that abnormality need not be a clonal genetic artifact as defined herein if the cell is not tested or subjected to an assay that would detect the genetic abnormality.

Also disclosed is a method and compositions for producing differentiated fetal cells. In the method, CD34+-enriched cells from maternal blood are incubated under conditions that promote differentiation of fetal CD34+ cells into or on one or more predetermined developmental pathways. It has been discovered that differentiated fetal cells have markers that distinguish the fetal cells from adult cells. For example, differentiated fetal CD34+ cells differ from the differentiated adult CD34+ cells in one or more cell markers. Differentiated fetal CD34+ cells can be distinguished from differentiated adult CD34+ cells by assessing one or more cell markers. Differentiated fetal CD34+ cells can be identified based on one or more cell markers. The cell surface marker can be CD1c, CD14, CD24, CD48, CD86, CD235a, MPO, MS4A6A, MS4A7, and ASGR2, or a combination. The presence or absence of these and other cell markers can be a function of growth and culture conditions (such as the presence and absence of particular cytokines and other media or growth factors and components). CD235a is also referred to as Glycophorin A. MPO is myeloperoxidase. Any other fetal markers can be used. Additional fetal markers can be identified, for example, using fetal marker identification techniques described in International Application No. WO 2005/123779 (Examples and Example 7 in particular), which is hereby incorporated by reference.

The conditions that promote differentiation of fetal CD34+ cells can include the presence of Stem Cell Factor (R&D Systems, Minneapolis, Minn.; Chemicon, Temecula, Calif.). In general, differentiation can involve culture of the cells in a culture medium such as HPGM (Cambrex, Walkersville, Md.) or Stemline II (Sigma-Aldrich, Milwaukee, Wis.) and in the presence or absence of other cytokines and growth factors such as IL-6, IL-3, EPO, TPO, Flt-1, Flt-3, IL-1, IL-11, GM-CSF, G-CSF, Wnt, Notch, IGF, BMP, Sonic Hedgehog, CxCL12, and basic fibroblast growth factor. As used herein, “differentiated cell” refers to cells one or more phenotypic characteristics of which has changed to a state more similar, or on the developmental pathway, to further differentiated cell types.

The disclosed method results from the discovery that fetal cells can be differentially expanded from maternal blood. Further, from populations of cells obtained by the disclosed method, it is possible to obtain pure cultures of fetal cells using known cloning and expansion techniques. The pure or enriched fetal cell populations obtained by the method have particular applications in preparing a cell therapy product including the fetal cells or cells derived from their differentiation. The disclosed method is non-invasive because a peripheral blood sample from a pregnant subject, not fetal blood, is used as the source of the fetal cells. The fetal cells are present in the peripheral blood of a pregnant subject. The disclosed method can be used to assess fetal characteristics (e.g. fetal sex and chromosomal abnormalities) or can be used to diagnose whether a fetus has a prenatal disease at an early stage of the gestational period. The non-invasive method of the present invention does not expose the fetus or mother to risks, e.g. infection, fetal injury, and miscarriage, associated with invasive methods such as amniocentesis.

Expansion and differentiation of fetal cells can be combined. For example, differentially expanded and/or enriched fetal and/or CD34+ cells can be differentiated into one or more predetermined developmental pathways. The fetal and/or CD34+ cells can be differentiated prior to, simultaneous with, or following expansion of the fetal CD34+ cells.

Expansion and/or differentiation of fetal cells can be combined with other preparation, isolation, sorting, selection and enrichment of fetal cells and/or CD34+ cells both as described herein and as known in the art. Useful combinations of this sort can include, for example, enrichment of CD34+ cells from maternal blood, differential expansion of fetal CD34+ cells relative to adult CD34+ cells, and isolation of the proportionally more numerous fetal CD34+ cells by marker-based cell sorting or separation. As another example, CD34+ cells can be enriched from maternal blood, fetal CD34+ cells can be differentiated into one or more predetermined developmental pathways, and the differentiated fetal CD34+ cells can be isolate by marker-based cell sorting or separation. These combinations of enrichment, differential expansion and/or differentiation, and separation produces a highly purified population of fetal cells. This can make any use of the fetal cells, such as analysis if the fetal cells, much more effective and efficient. Cell sorting and separation can be based, for example, on the presence and/or absence of one or more particular cell markers. Any suitable cell surface markers can be used. Useful cell surface markers include CD1c, CD14, CD24, CD48, CD86, CD235a, MPO, MS4A6A, MS4A7, and ASGR2. These cell markers are present on fetal cells. As used herein, “enrichment” refers to an increase in the proportion of one or more compositions or cells in a sample or mixture. Enrichment can be accomplished by, for example, gathering or collecting the compositions or cells to be enriched (positive selection), removing or depleting compositions or cells not to be enriched, or a combination. As used herein, “depletion” refers to a decrease in the proportion of one or more compositions or cells in a sample or mixture. Depletion can be accomplished by, for example, removing compositions or cells (including by killing the cells) to be depleted, gathering or collecting the compositions or cells that are not to be depleted, or a combination.

Fetal cells and CD34+ cells can be enriched from maternal blood. For example, fetal cells can be enriched from maternal blood by selecting or sorting cells based on the presence or absence of the markers CD34, CD133, CD117, CD2, and/or CD90, by removing or lysing red blood cells, by selecting or sorting cells based on the presence or absence one or more of the markers, or a combination. CD117 is also known as SCF receptor and CD2 is a T cell marker. Many techniques for sorting and separating cells based on the presence and/or absence of cell markers are known and can be used in the disclosed method. Any cell marker can be used, including cell surface markers and internal markers. For example, fetal cells can be enriched from maternal blood by immunomagnetic selection (magnetic activated cell sorting (MACS), for example), fluorescence activated cell sorting (FACS), and similar techniques. Fetal cells can be enriched from maternal blood by positive selection of fetal cells, by direct selection of fetal cells, by indirect selection of fetal cells, by depletion of non-fetal cells, or by a combination. CD34+ cells can be enriched from maternal blood by positive selection of CD34+ cells, by direct selection of CD34+ cells, by indirect selection of CD34+ cells, by depletion of non-CD34+ cells, by depletion of CD34− cells, or by a combination. Fetal and/or CD34+ cells can be enriched from maternal blood by selecting or sorting cells based on the presence or absence of one or more fetal cell markers. The fetal cell markers can be CD1c, CD14, CD24, CD48, CD86, CD235a, MPO, MS4A6A, MS4A7, and ASGR2 or a combination of these markers. Fetal and/or CD34+ cells can be enriched from maternal blood by positive selection of CD34+ cells and by depletion of CD38+ cells and GlycophorinA+ cells. This generally can be done prior to expansion of the cells.

Also disclosed are fetal cells made or obtained using the disclosed methods. For example, disclosed are expanded and/or differentiated fetal cells. Fetal cells can be obtained, for example, by incubating cells from a sample of maternal blood containing CD34+ cells of both maternal and fetal origin are incubated in the presence of SCF in serum free media. Fetal cells can also be obtained by incubating CD34+-enriched cells from maternal blood in the presence of SCF and HPGM. Differentiated fetal cells can be obtained, for example, by incubating CD34+-enriched cells from maternal blood under conditions that promote differentiation of fetal CD34+ cells into one or more predetermined developmental pathways, wherein conditions that promote differentiation of fetal CD34+ cells include the presence of Stem Cell Factor and identifying differentiated fetal CD34+ cells based on one or more cell markers, wherein the cell surface marker is CD1c, CD14, CD24, CD48, CD86, CD235a, MPO, MS4A6A, MS4A7, and ASGR2, or a combination. In general, differentiation can involve culture of the cells in a culture medium such as HPGM (Cambrex, Walkersville, Md.) or Stemline H (Sigma-Aldrich, Milwaukee, Wis.), or equivalent, and in the presence or absence of other cytokines and growth factors such as IL-6, IL-3, EPO, TPO, Flt-1, Flt-3, IL-1, IL-11, GM-CSF, G-CSF, Wnt, Notch, IGF, BMP, Sonic Hedgehog, CxCL12, and basic fibroblast growth factor. Also disclosed are compositions that includes a mixture of fetal and maternal stem cells wherein the fetal cells are present at a concentration of greater than 5 times that of the maternal cells. Also disclosed are compositions that includes a mixture of fetal and maternal stem cells wherein the fetal cells are present at a concentration of greater than 3 times that of the maternal cells.

The disclosed fetal cells can be used for any purpose and in any way that fetal cells can be used. The disclosed fetal cells are particularly useful for analyzing one or more characteristics of the fetal cells relevant to the heath, condition and prognosis of a gestating fetus. Any characteristic can be analyzed, such as genetic, physiological, chromosomal, genomic, proteomal, biochemical, and other cellular characteristics. Methods, techniques, assays and systems for such analysis are known and can be used with the disclosed fetal cells. The disclosed fetal cells can also be cultured, stored, differentiated, transformed, transfected, and used for testing, assays, production of biologicals, chemicals, and cellular components.

Detection and/or analysis of characteristics of fetal cells is a preferred use for the disclosed fetal cells. Thus, disclosed is a method of analyzing one or more of the fetal cells for one or more characteristics. The fetal cells can be fetal cells obtained, expanded and/or differentiated as described herein. The fetal cells can form colonies and one or more colonies of fetal cells can be harvested, where one or more of the expanded fetal CD34+ cells that are analyzed are derived from one or more of the harvested colonies.

The characteristic(s) to be detected or analyzed can be any characteristic of the fetal cells. Numerous characteristics of cells are known, and any such characteristics can be analyzed in the disclosed fetal cells. For example, the characteristic can be genotype, phenotype, physiological function, biochemical function, or a combination. The characteristic can be the presence or absence of one or more particular nucleic acid sequences, or the presence or absence of particular mutations, alternative sequences, alleles, homologous sequence, and the like. The characteristic can be the sex of the fetus from which the fetal cells derived. The sex of the fetus can be analyzed, for example, by detecting the presence of Y chromosomes, X chromosomes, or both in the fetal cells.

The characteristic can be a disease or condition or an indicator of a disease or condition. The indicator of the disease or condition can be analyzed by detecting one or more mutations, single nucleotide polymorphisms, genetic markers, or a combination associated with the disease or condition. The mutation, single nucleotide polymorphism, or genetic marker can be, for example, a cystic fibrosis-associated mutation, single nucleotide polymorphism, or genetic marker, a Duchenne muscular dystrophy-associated mutation, single nucleotide polymorphism, or genetic marker, a hemophilia A-associated mutation, single nucleotide polymorphism, or genetic marker, a Gaucher disease-associated mutation, single nucleotide polymorphism, or genetic marker, a sickle cell anemia-associated mutation, single nucleotide polymorphism, or genetic marker, a Tay-Sachs-associated mutation, single nucleotide polymorphism, or genetic marker, or a combination.

The characteristic can be a chromosomal abnormality. The chromosomal abnormality can be chromosomal aneuploidy, chromosomal translocation, deletion, duplication or a combination. The chromosomal aneuploidy can be trisomy 21, trisomy 18, trisomy 13 or a combination.

Numerous tests, assays, and techniques are known for detecting or analyzing cell characteristics, and such tests, assays and techniques can be used to analyze the disclosed fetal cells.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a fetal CD34+ cell is disclosed and discussed and a number of manipulations and modifications that can be made to a number of cells including the fetal CD34+ cell are discussed, each and every combination and permutation of cells and the manipulations and modifications that are possible are specifically contemplated unless specifically indicated to the contrary. Thus, if a class of cells A, B, and C are disclosed as well as a class of manipulations D, E, and F and an example of a combination A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Cell Culture Medium

The disclosed method of expanding and/or differentiating fetal cells involves incubation and culturing of cells and the use of culture medium. Any suitable culture medium can be used. As disclosed herein, differential expansion of fetal cells makes use of Stem Cell Factor. Thus, the cell culture medium can be any suitable base medium further including SCF. The culture medium can also include other cytokines such as IL-3 and IL-6. The culture medium can include SCF at a concentration of from about 15, 20, 25, 30, 35, 40, 45, or 50 ng/ml to about 12.5, 25, 50, 55, 60, 65, 70, 75, 80, 85, 90, 95, 100, 105, 110, 115, 120, 125, 130, 135, 140, 145, 150, 200, or 250 ng/ml. The culture medium can include SCF at a concentration of about 12.5, 15, 20, 25, 30, 35, 40, 45, 50, 55, 60, 65, 70, 75, 80, 85, 90, 95, 100, 105, 110, 115, 120, 125, 130, 135, 140, 145, or 150 ng/ml. The culture medium can include IL-6 at a concentration of from about 1, 2, 3, 4, 5, 6, 7, 8, 9, or 10 ng/ml to about 11, 12, 13, 14, 15, 16, 17, 18, 19, 20, 21, 22, 23, 24, or 25 ng/ml. The culture medium can include IL-6 at a concentration of about 1, 2, 3, 4, 5, 6, 7, 8, 9, 10, 11, 12, 13, 14, 15, 16, 17, 18, 19, 20, 21, 22, 23, 24, or 25 ng/ml. The culture medium can include IL-3 at a concentration of from about 1, 2, 3, 4, 5, 6, 7, 8, 9, 10, 11, 12, 13, 14, or 15 ng/ml to about 16, 17, 18, 19, 20, 21, 22, 23, 24, 25, 26, 27, 28, 29, or 30 ng/ml. The culture medium can include IL-3 at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml. The culture medium can include SCF at a concentration of from about 50 ng/ml, IL-6 at a concentration of about 5 ng/ml, and IL-3 at a concentration of about 10 ng/ml.

The culture medium can also include other factors, such as IL-6, IL-3, EPO, TPO, Flt-1, Flt-3, IL-1, GM-CSF, G-CSF, Wnt, Notch, IGF, BMP, Sonic Hedgehog, CxCL12, basic fibroblast growth factor, specific vitamins, and specific antibodies capable of inhibiting adult cell growth. Such other factors can also be absent. For example, the culture medium can lack IL-3, IL-6, TPO or. EPO. The medium can also be supplemented with one or more additional cytokines at a concentration from about 0.1 ng/mL to about 500 ng mL, more usually 10 ng/mL to 100 ng/mL. Suitable cytokines, include but are not limited to, IL-6, G-CSF, IL-3, GM-CSF, IL-1α, IL-11 MIP-1α, LIF, c-mpl ligand/TPO, and flk2/flk3 ligand. (Nicola, et al., Blood 54:614-627, 1979; Golde et al., Proc. Natl. Acad. Sci. (USA) 77, 593-596, 1980; Lusis, Blood 57, 13-21, 1981; Abboud et al., Blood 58, 1148-1154, 1981; Okabe, Cell. Phys., 110, 43-49, 1982; Fauser et al., Stem Cells, 1, 73-80, 1981). The culture can include at least IL-3 and IL-6. The culture can include one or more of c-kit ligand, IL-6, IL-3, EPO, TPO, Flt-1, Flt-3, IL-1, GM-CSF, G-CSF, Wnt, Notch, IGF, BMP, Sonic Hedgehog, CxCL12, basic fibroblast growth factor or specific vitamins or specific antibodies capable of inhibiting adult cell growth.

The base medium can be any medium suitable for growing stem cells. For example, the base medium can be Cambrex's Hematopoietic Progenitor Growth Medium (HPGM), Dulbecco's modified Eagle's medium (DMEM), IMDM and RPMI-1640, Knockout DMEM (Invitrogen), and Stemline II medium (Sigma-Aldrich). The medium can contain retinoic acid and essential vitamins. The medium can contain about 5%, 10%, 15%, 20% serum or serum replacements (e.g. knockout serum replacement; Invitrogen). In one aspect, the serum does not contain non-human animal products. In another aspect, the serum is human serum. In another aspect, the medium can be a serum-free defined medium. An example of the ingredients of a defined medium are provided in Table 1.

TABLE 1

| | | | |
|---|---|---|---|
| | Molecular | Concentration | Molarity |
| COMPONENTS | Weight | (mg/L) | (mM) |

Amino Acids

| | | | |
|---|---|---|---|
| Glycine | 75 | 30 | 0.400 |
| L-Alanine | 89 | 25 | 0.281 |
| L-Arginine hydrochloride | 211 | 84 | 0.398 |
| L-Asparagine (freebase) | 150 | 25 | 0.167 |
| L-Aspartic acid | 133 | 30 | 0.226 |
| L-Cystine 2HCl | 240 | 91.4 | 0.381 |
| L-Glutamic Acid | 147 | 75 | 0.510 |
| L-Glutamine | 146 | 584 | 4.00 |
| L-Histidine | 210 | 42 | 0.200 |
| hydrochloride-H₂O | | | |
| L-Isoleucine | 131 | 105 | 0.802 |
| L-Leucine | 131 | 105 | 0.802 |
| L-Lysine hydrochloride | 183 | 146 | 0.798 |
| L-Methionine | 149 | 30 | 0.201 |
| L-Phenylalanine | 165 | 66 | 0.400 |
| L-Proline | 115 | 40 | 0.348 |
| L-Serine | 105 | 42 | 0.400 |
| L-Threonine | 119 | 95 | 0.798 |
| L-Tryptophan | 204 | 16 | 0.0784 |
| L-Tyrosine disodium salt | 225 | 104 | 0.462 |
| L-Valine | 117 | 94 | 0.803 |

Vitamins

| | | | |
|---|---|---|---|
| Biotin | 244 | 0.013 | 0.0000533 |
| Choline chloride | 140 | 4 | 0.0286 |
| D-Calcium pantothenate | 477 | 4 | 0.00839 |
| Folic Acid | 441 | 4 | 0.00907 |
| i-Inositol | 180 | 7.2 | 0.0400 |
| Niacinamide | 122 | 4 | 0.0328 |
| Pyridoxal hydrochloride | 204 | 4 | 0.0196 |
| Riboflavin | 376 | 0.4 | 0.00106 |
| Thiamine hydrochloride | 337 | 4 | 0.0119 |
| Vitamin B12 | 1355 | 0.013 | 0.0000096 |

Inorganic Salts

| | | | |
|---|---|---|---|
| Calcium Chloride (CaCl₂) | 111 | 165 | 1.49 |
| (anhyd.) | | | |
| Magnesium Sulfate (MgSO₄) | 120 | 97.67 | 0.814 |
| (anhyd.) | | | |
| Potassium Chloride (KCl) | 75 | 330 | 4.40 |
| Potassium Nitrate (KNO₃) | 101 | 0.076 | 0.000752 |
| Sodium Bicarbonate | 84 | 3024 | 36.00 |
| (NaHCO₃) | | | |
| Sodium Chloride (NaCl) | 58 | 4500 | 77.59 |
| Sodium Phosphate monobasic | 138 | 125 | 0.906 |
| (NaH₂PO₄—H₂O) | | | |
| Sodium Selenite | 263 | 0.0173 | 0.0000658 |
| (Na2SeO3—5H20) | | | |

Other Components

| | | | |
|---|---|---|---|
| D-Glucose (Dextrose) | 180 | 4500 | 25.00 |
| HEPES | 238 | 5958 | 25.03 |
| Phenol Red | 376.4 | 15 | 0.0399 |
| Sodium Pyruvate | 110 | 110 | 1.000 |

### REFERENCES

Dulbecco, R. and Freeman, G. (1959) Virology 8:396.

Smith, J. D., Freeman, G., Vogt, M., et al., (1960) Virology 12:185.

Iscove, N. N. and Melchers, F. (1978) J. Exper. Med., 147:923.

It should be recognized that SCF and other cytokines are proteins and as such certain modifications can be made to the proteins which are silent and do not remove the activity of the proteins as described herein. Such modifications include additions, substitutions and deletions. Methods modifying proteins are well established in the art (Sambrook et al., Molecular Cloning: A Laboratory Manual, 2nd Ed., Cold Spring Harbor Laboratory, Cold Spring Harbor, N.Y., 1989).

For example, 1 liter of the cell culture medium can include HPGM, about 50 ng per ml SCF, about 1 mM glutamine, about 0.1 M mercaptoethanol, and about 0.1 mM non-essential amino acids. Alternatively, the medium can include effective amounts of at least one of a peptone, a protease inhibitor and a pituitary extract and effective amounts of at least one of human serum albumin or plasma protein fraction, heparin, a reducing agent, insulin, transferrin and ethanolamine. Other suitable media formulations are well known to those of skill in the art, see for example, U.S. Pat. No. 5,728,581. Other ingredients and modifications that can be made to the provided medium that are suitable for culturing stem cells are known in the art and are contemplated herein.

1. Stem Cell Factor

The cell culture medium can include Stem Cell Factor (SCF), including human SCF sufficient to support differential expansion of fetal cells. For example, the culture medium can include SCF at a concentration of from about 15, 20, 25, 30, 35, 40, 45, or 50 ng/ml to about 12.5, 25, 50, 55, 60, 65, 70, 75, 80, 85, 90, 95, 100, 105, 110, 115, 120, 125, 130, 135, 140, 145, 150, 200, or 250 ng/ml. The culture medium can include SCF at a concentration of about 12.5, 15, 20, 25, 30, 35, 40, 45, 50, 55, 60, 65, 70, 75, 80, 85, 90, 95, 100, 105, 110, 115, 120, 125, 130, 135, 140, 145, or 150 ng/ml. SCF is also called Steel factor, mast cell growth factor and c-kit ligand in the art. SCF is a transmembrane protein with a cytoplasmic domain and an extracellular domain. SCF is well known in the art; see European Patent Publication No. 0 423 980 A1, corresponding to European Application No. 90310889.1.

The purification, cloning and use of SCF have been reported in U.S. Pat. No. 6,204,363, which is incorporated herein by reference in its entirety for this teaching. SCF, as used herein, includes natural forms, including such forms produced in mammals, such as humans, as well as homologues and mutants thereof. SCF can be obtained by any method, and includes the use of modified or truncated SCF molecules and SCF analogs which retain the desired activity. The nucleic acid sequence for human stem cell factor (SCF) can be found at GenBank Accession No. NM_{—}000899 and the corresponding amino acid sequence can be found at Accession No. NP_{—}000890. For example, SCF for use in the herein disclosed compositions and methods can include a polypeptide having at least 70, 75, 80, 85, 90, 95, 100% sequence identity to the amino acid sequence set forth in Accession No. NP_{—}000890.

SCF may be obtained by techniques well known in the art from a variety of cell sources which synthesize bioactive SCF including, for example, cells which naturally produce SCF and cells transfected with recombinant DNA molecules capable of directing the synthesis and/or secretion of SCF. Alternatively, SCF may be synthesized by chemical synthetic methods including but not limited to solid phase peptide synthesis.

### B. Labels and Labeled Molecules

To aid in detection, sorting and separation of cells, labels can be associated with cells. For example, antibodies specific for cell markers can be labeled. As used herein, a label is any molecule that can be associated with a cell, directly or indirectly, and which results in a measurable, detectable signal, either directly or indirectly. Many such labels are known to those of skill in the art. Examples of labels suitable for use in the disclosed method are radioactive isotopes, fluorescent molecules, phosphorescent molecules, enzymes, antibodies, and ligands. Fluorescent labels are particularly useful for cell detection, sorting and separation.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin,

Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC. Any other suitable labels can be used, such a Hoechst dyes and quantum dots.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 run), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Molecules that combine two or more of these labels are also considered labels. Any of the known labels can be used with the disclosed methods and cells. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody.

Labeled antibodies are useful with the disclosed method. Such antibodies can be used to label, sort and/or separate cells to which the antibodies can bind. Useful antibodies are antibodies directed against the cell proteins CD34, CD1c, CD14, CD24, CD48, CD86, CD235a, MPO, MS4A6A, MS4A7, ASGR2, CD34, CD133, CD117, CD2, or CD90. Antibodies to these and other markers are known and can be obtained commercially. For example, many useful antibodies to markers can be obtained from BD Bioscience, San Jose, Calif., Sigma-Aldrich, Milwaukee, Wis., and Vector Labs, Burlingame, Calif.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for expanding fetal cells, the kit including SCF and HPGM. The kits also can contain antibodies for cell markers.

### D. Mixtures

Disclosed are mixtures formed by performing or preparing to perform the disclosed method. For example, disclosed are mixtures including fetal and maternal cells where the fetal cells are enriched 5 fold or more relative to the maternal cells. Also disclosed are mixtures including fetal and maternal cells where the fetal cells are enriched 3 fold or more relative to the maternal cells.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. Also, whenever the method alters the condition and/or ratio of one or more compositions or components, performing the method results in a mixture of the compositions and components as altered. The present disclosure contemplates these mixtures, obtained by the performance of the disclosed methods as well as mixtures containing any disclosed reagent, composition, or component, for example, disclosed herein.

### E. Systems

Disclosed are systems useful for performing, or aiding in the performance of, the disclosed method. Systems generally include combinations of articles of manufacture such as structures, machines, devices, and the like, and compositions, compounds, materials, and the like. Such combinations that are disclosed or that are apparent from the disclosure are contemplated. For example, disclosed and contemplated are systems including columns and cells; cell sorters and cells; columns, cell sorters and cells; cell culture apparatus and cells; columns, cell culture apparatus and cells; and columns, cell culture apparatus, cell sorters and cells.

### F. Data Structures and Computer Control

Disclosed are data structures used in, generated by, or generated from, the disclosed method. Data structures generally are any form of data, information, and/or objects collected, organized, stored, and/or embodied in a composition or medium. Fetal cell analysis results stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, analysis of fetal cells. Other uses include assessment and diagnosis of prenatal conditions and status. Other uses are disclosed, apparent from the disclosure, and/or will be understood by those in the art.

### Methods

Disclosed is a method for the differential expansion of fetal cells over maternal cells. In the method, cells from a sample of maternal blood containing CD34+ cells of both maternal and fetal origin are incubated in the presence of Stem Cell Factor (SCF) in serum free media. It has been discovered that incubation of fetal cells in the presence of SCF will preferentially expand relative to adult cells. Fetal cells can also be identified, enriched or obtained by differential expansion of the fetal cells during colony formation. It has been discovered that differential expansion of fetal cells can result in colonies of fetal cells that are larger than colonies of adult cells. The fetal CD34+ cells can be expanded in the absence of significant expansion of adult cells. The fetal CD34+ cells can be expanded without generation of significant clonal genetic artifacts during expansion. Also disclosed is a method for producing differentiated fetal cells. It has been discovered that differentiated fetal cells have markers that distinguish the fetal cells from adult cells.

The fetal cell sample which is produced by the disclosed method is one in which the proportion of fetal cells present in the sample is greatly increased compared to the proportion of fetal cells present in the original maternal blood sample. Thus, the resultant fetal cell sample is one which is highly enriched in fetal cells. This enrichment for fetal cells is sufficient to allow for analysis of fetal cells which otherwise could not be analyzed in the unenriched, original blood sample.

The differentially expanded cells can be CD34+ cells. The fetal cells can be differentially expanded by a factor of at least about 2, 3, 4, 5, 6, 7, 8, 9, 10, 11, 12, 13, 14, 15, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, or 200 compared with maternal cells. The cells from maternal blood can be incubated in the presence of SCF at a concentration of from about 15, 20, 25, 30, 35, 40, 45, or 50 ng/ml to about 12.5, 25, 50, 55, 60, 65, 70, 75, 80, 85, 90, 95, 100, 105, 110, 115, 120, 125, 130, 135, 140, 145, 150, 200, or 250 ng/ml. The cells from maternal blood can be incubated in the presence of SCF at a concentration of about 12.5, 15, 20, 25, 30, 35, 40, 45, 50, 55, 60, 65, 70, 75, 80, 85, 90, 95, 100, 105, 110, 115, 120, 125, 130, 135, 140, 145, or 150 ng/ml.

The cells from maternal blood can be incubated in the presence of IL-6. The cells from maternal blood can be incubated in the presence of IL-6 at a concentration of from about 1, 2, 3, 4, 5, 6, 7, 8, 9, or 10 ng/ml to about 11, 12, 13, 14, 15, 16, 17, 18, 19, 20, 21, 22, 23, 24, or 25 ng/ml. The cells from maternal blood can be incubated in the presence of IL-6 at a concentration of about 1, 2, 3, 4, 5, 6, 7, 8, 9, 10, 11, 12, 13, 14, 15, 16, 17, 18, 19, 20, 21, 22, 23, 24, or 25 ng/ml.

The cells from maternal blood can be incubated in the presence of IL-3. The cells from maternal blood can be incubated in the presence of IL-3 at a concentration of from about 1, 2, 3, 4, 5, 6, 7, 8, 9, 10, 11, 12, 13, 14, or 15 ng/ml to about 16, 17, 18, 19, 20, 21, 22, 23, 24, 25, 26, 27, 28, 29, or 30 ng/ml. The cells from maternal blood can be incubated in the presence of IL-3 at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/ml.

The cells from maternal blood can be incubated in the presence of SCF at a concentration of from about 50 ng/ml, IL-6 at a concentration of about 5 ng/ml, and IL-3 at a concentration of about 10 ng/ml.

The cells from maternal blood can be incubated in the presence of IL-3 and IL-6. The cells from maternal blood can be incubated in the absence of IL-3, IL-6, TPO and/or EPO. The cells from maternal blood can be incubated in the presence of SCF at a concentration of from about 100 ng/ml.

The fetal CD34+ cells can be expanded in the absence of significant or substantial expansion of adult cells. By significant expansion is meant that the cells do not expand by more than 10%. By substantial expansion is meant that the cells do not expand by more than 50%. Percent expansion refers to the number of cells present after expansion expressed as a percentage of the starting number of cells. Thus, 10% expansion would result in a number of cells 110% (or 1.1 times) the number of cells at the start. The fetal CD34+ cells can be expanded in the absence of expansion of adult cells by, for example, more than 1%, 2%, 3%, 4%, 5%, 6%, 7%, 8%, 9%, 10%, 15%, 20%, 25%, 30%, 35%, 40%, 45%, 50%, 55%, 60%, or 65%. The adult cells can be expanded by, for example, less than 1%, 2%, 3%, 4%, 5%, 6%, 7%, 8%, 9%, 10%, 15%, 20%, 25%, 30%, 35%, 40%, 45%, 50%, 55%, 60%, or 65% during expansion of the fetal CD34+ cells.

In some forms of the method, a sample of maternal blood is removed early on in pregnancy (for example at about the fifth week of a human pregnancy). However, removing the sample of maternal blood and the expansion of fetal cells can be carried out at any time from the start to the end of pregnancy. For example, sampling and expansion of fetal cells can be carried out between the 7th and 15th week of pregnancy or between the 10th and 15th week of pregnancy.

Because the disclosed method involves expansion of fetal cells, it is not necessary to have a large sample of maternal blood. Nevertheless, in general, between 3 and 20 milliliters of maternal blood can be removed, preferably between 5 and 10 milliliters. In order to increase the sensitivity of the diagnosis, it is possible to take a plurality of independent samples to repeat the diagnosis on different independent samples.

Fetal origin of cells can be confirmed, if desired. This can be accomplished using known markers and techniques. For example, U.S. Patent Application Publication 20050049793 described some useful techniques.

### A. Expansion of Fetal Cells

The disclosed expansion method generally requires inoculating the population of maternal blood cells into an expansion container and in a volume of a suitable medium such that the cell density is from at least about 5,000, preferably 10,000 to about 1,000,000 cells/mL of medium, and more preferably from about 10,000 to about 500,000 cells/mL of medium, and at an initial carbon dioxide concentration of from about 2 to 20% and preferably less than 8%. In some forms of the method, the initial oxygen concentration is in a range from about 2% to about 6%. In some forms of the method, the inoculating population of cells is enriched in CD34+ cells and is from about 7,000 cells/mL to about 20,000 cells/mL and preferably about 10,000 cell/mL. In some forms of the method, the inoculation population of cells is derived from mobilized peripheral blood and is from about 20,000 cells/mL to about 1,000,000 cells/mL, preferably 500,000 cells/mL.

Any suitable expansion container, flask, or appropriate tube such as a 24 well plate, 12.5 cm² T flask or gas-permeable bag can be used in the disclosed method. Such culture-containers are commercially available from Falcon, Corning or Costor. As used herein, “expanion container” also is intended to include any chamber or container for expanding cells whether or not free standing or incorporated into an expansion apparatus such as bioreactors. In one embodiment, the expansion container is a reduced volume space of the chamber which is formed by a depressed surface and a plane in which a remaining cell support surface is orientated.

Various media can be used for the expansion of fetal cells. Illustrative media include Cambrex's HPGM, Dulbecco's MEM, IMDM and RPMI-1640, and Sigma-Aldrich's Stemline II medium that can be supplemented with a variety of different nutrients, growth factors, cytokines, etc. The media can be serum free or supplemented with suitable amounts of serum such as fetal calf serum or autologous serum. Preferably, the medium is serum-free or supplemented with autologous serum. For example, 1 liter of the cell culture medium can include HPGM, about 50 ng per ml SCF, about 1 mM glutamine, about 0.1 M mercaptoethanol, and about 0.1 mM non-essential amino acids. Alternatively, the medium can include effective amounts of at least one of a peptone, a protease inhibitor and a pituitary extract and effective amounts of at least one of human serum albumin or plasma protein fraction, heparin, a reducing agent, insulin, transferrin and ethanolamine. Other suitable media formulations are well known to those of skill in the art, see for example, U.S. Pat. No. 5,728,581. The medium can lack and/or not be supplemented with serum.

Regardless of the specific medium being used, the medium will include an effective amount of SCF. The medium can also be supplemented, or not supplemented, with one or more additional cytokines at a concentration from about 0.1 ng/mL to about 500 ng mL, more usually 10 ng/mL to 100 ng/mL. Suitable cytokines, include but are not limited to IL-6, G-CSF, IL-3, GM-CSF, IL-1α, IL-11 MIP-1α, LIF, c-mpl ligand/TPO, and flk2/flk3 ligand. (Nicola, et al., Blood 54:614-627, 1979; Golde et al., Proc. Natl. Acad. Sci. (USA) 77, 593-596, 1980; Lusis, Blood 57; 13-21, 1981; Abboud et al., Blood 58, 1148-1154, 1981; Okabe, J. Cell. Phys., 110, 43-49, 1982; Fauser et al., Stem Cells, 1, 73-80, 1981). The culture can include at least IL-3 and IL-6. The culture can include one or more of c-kit ligand, IL-6, IL-3, EPO, TPO, Flt-1, Flt-3, IL-1, GM-CSF, G-CSF, Wnt, Notch, IGF, BMP, Sonic Hedgehog, CxCL12, basic fibroblast growth factor or specific vitamins or specific antibodies capable of inhibiting adult cell growth. In one embodiment, the cytokines are contained in the media and replenished by media perfusion. Alternatively, when using a bioreactor system, the cytokines may be added separately, without media perfusion, as a concentrated solution through separate inlet ports. When cytokines are added without perfusion, they will typically be added as a 10× to 100× solution in an amount equal to 1/10 to 1/100 of the volume in the bioreactors with fresh cytokines being added approximately every 2 to 4 days. Further, fresh concentrated cytokines also can be added separately in addition, to cytokines in the perfused media.

Any cytokines, growth factors or media components other than SCF can be specifically included or excluded from the culture. For example, a cytokine, growth factor or component can be present, absent, included or not included in the culture or growth medium, or the culture or growth medium can be supplemented or not supplemented with a cytokine, growth factor or component. It is specifically contemplated and disclosed herein that such presence, absence, inclusion, exclusion, supplementation and lack of supplementation can apply in any and all combinations to every different cytokine, growth factor and/or component disclosed herein. For example, the combination of Flt-3 ligand and TPO can be excluded, the combination of IL-3 and IL-6 can be excluded, the combination of EPO and TPO can be excluded, and/or EPO can be excluded.

The cells can be cultured under suitable conditions such that the cells condition the medium. Improved expansion of fetal cells may be achieved when the culture medium is not changed, e.g., perfusion does not start until after the first several days of culture.

In most aspects, suitable conditions include culturing at 33 to 39, and preferably around 37° C. (the initial oxygen concentration is preferably 2-8%, and most preferably, about 5%) for at least 6 days and preferably from about 7 to about 10 days, to allow release of autocrine factors from the cells without release of sufficient waste products to substantially inhibit fetal cell expansion. After that time, the oxygen concentration is preferably increased to about 20%, either stepwise or gradually over the remainder of the culture period. Preferably, fetal cells will be grown for around 5-14 days.

After the initial culture period without medium exchange, the culture medium can be exchanged at a rate which allows expansion of the fetal cells. In a system where no variable volume is used, medium can be exchanged on or between day 7 and day 10. The exchange of fresh medium in a perfused system can be laminar. This uniform, nonturbulent, flow prevents the formation of “dead spaces” where patches of cells are not exposed to medium. The medium can be exchanged at a rate of from about 0.10/day to 0.50/day or 1/10 to ½ volume exchange per day. Preferably, the perfusion rate can be from about 0.25/day to 0.40/day. Most preferably, perfusion can be at a rate of 0.27/day starting around day 14, and for mobilized peripheral blood stem cells, perfusion starts at 0.25/day around day 10 and increases to 0.40/day around day 12.

Preferably, the cell concentration is kept at an optimum throughout expansion. For instance, fetal cells can expand up to about 100 fold compared to maternal cells. Fetal cells have a large proliferative capacity, as such, where culture is performed in a closed system such a system must provide enough volume for total cell expansion. Cells can be expanded in a bioreactor such as the one described in U.S. Pat. No. 5,728,581. The shape of the device allows the medium volume to be increased up to three-fold without significantly reducing the oxygen transfer efficiency to the cells.

### B. Fetal Cell Differentiation

Fetal cells can be differentiated using any suitable conditions. For example, fetal cells can become differentiated during expansion, and thus the conditions for fetal cell differentiation can be the same as those used for fetal cell expansion. For example, fetal cells can be differentiated by culturing the cells in the presence of Stem Cell Factor (R&D Systems, Minneapolis, Minn.; Chemicon, Temecula, Calif.). In general, differentiation can involve culture of the cells in a culture medium such as HPGM (Cambrex, Walkersville, Md.) or Stemline II (Sigma-Aldrich, Milwaukee, Wis.) and in the presence or absence of other cytokines and growth factors such as IL-6, IL-3, EPO, TPO, Flt-1, Flt-3, IL-1, IL-11, GM-CSF, G-CSF, Wnt, Notch, IGF, BMP, Sonic Hedgehog, CxCL12, and basic fibroblast growth factor. Other culture conditions and factors as described elsewhere herein can also be used for differentiation of fetal cells. Culture of the fetal cells results in changes in cell markers and may result in changes in morphology or other phenotypes.

### C. Enrichment

Expansion and/or differentiation of fetal cells can be combined with other preparation, isolation, sorting, selection and enrichment of fetal cells and/or CD34+ cells both as described herein and as known in the art. Useful combinations of this sort can include, for example, enrichment of CD34+ cells from maternal blood, differential expansion of fetal CD34+ cells relative to adult CD34+ cells, and isolation of the proportionally more numerous fetal CD34+ cells by marker-based cell sorting or separation. As another example, CD34+ cells can be enriched from maternal blood, fetal CD34+ cells can be differentiated into one or more predetermined developmental pathways, and the differentiated fetal CD34+ cells can be isolate by marker-based cell sorting or separation. Cell sorting and separation can be based, for example, on the presence and/or absence of one or more particular cell markers, such as cell surface markers. Any suitable cell markers can be used. Useful cell markers include CD1c, CD14, CD24, CD48, CD86, CD235a, MPO, MS4A6A, MS4A7, and ASGR2. These cell markers are present on fetal cells. As used herein, “enrichment” refers to an increase in the proportion of one or more compositions or cells in a sample or mixture. Enrichment can be accomplished by, for example, gathering or collecting the compositions or cells to be enriched (positive selection), removing or depleting compositions or cells not to be enriched, or a combination.

Fetal cells and CD34+ cells can be enriched from maternal blood. For example, fetal cells can be enriched from maternal blood by selecting or sorting cells based on the presence or absence of the markers CD34, CD133, CD117, CD2, or CD90, by removing or lysing red blood cells, by selecting or sorting cells based on the presence or absence one or more of the markers, or a combination. Many techniques for sorting and separating cells based on the presence and/or absence of cell markers are known and can be used in the disclosed method. For example, fetal cells can be enriched from maternal blood by immunomagnetic selection, fluorescence activated cell sorting (FACS), and similar techniques. Fetal cells can be enriched from maternal blood by positive selection of fetal cells, by direct selection of fetal cells, by indirect selection of fetal cells, by depletion of non-fetal cells, or by a combination. CD34+ cells can be enriched from maternal blood by positive selection of CD34+ cells, by direct selection of CD34+ cells, by indirect selection of CD34+ cells, by depletion of non-CD34+ cells, by depletion of CD34− cells, or by a combination. Fetal and/or CD34+ cells can be enriched from maternal blood by selecting or sorting cells based on the presence or absence of one or more fetal cell markers. The fetal cell markers can be CD1c, CD14, CD24, CD48, CD86, CD235a, MPO, MS4A6A, MS4A7, and ASGR2 or a combination of these markers. Fetal and/or CD34+ cells can be enriched from maternal blood by positive selection of CD34+ cells and by depletion of CD38+ cells and GlycophorinA+ cells. This generally can be done prior to expansion of the cells.

One or more monoclonal antibodies which are specific for maternal cells (that is, which recognize and bind to a cell marker present on maternal cells, e.g. maternal leukocytes, but not present on fetal cells) can be used to facilitate removal of maternal cells from the sample of maternal blood, thereby separating fetal cells from maternal cells and resulting in an enrichment of fetal cells in the cell population from which the maternal cells were removed. For example, a monoclonal antibody HLe-1 (Becton-Dickinson Monoclonal Center, Mountain View, Calif., catalog #7463) recognizes and binds to an antigen present on mature human leukocytes and can be used to remove maternal cells from a maternal blood sample. See PCT Publication WO 91/07660. A monoclonal antibody which recognizes and binds to maternal cells but not fetal cells can be combined with a monoclonal antibody which recognizes and binds fetal cells but not maternal cells in order to both remove maternal cells and to facilitate enrichment for fetal granulocytes.

Maternal cells can be depleted prior to fetal cell expansion and/or differentiation. The mononuclear cell layer can be initially isolated from a maternal blood sample, for example following Ficoll-Hypaque density gradient centrifugation. The resulting cell suspension consists predominantly of maternal cells. In order to increase the eventual proportion of fetal cells present thereby enriching for fetal cells, maternal cells are selectively removed by incubating the cells with antibodies which recognize and bind maternal cells and which are attached to a solid support. Such supports can include magnetic beads, plastic flasks, plastic dishes and columns. The antibodies recognize and bind antigens present on the maternal cells, e.g. an antibody specific for an antigen present on human mature leukocytes can be used. Thus, a majority of maternal cells are eliminated by virtue of being bound to the solid support. The total number of cells remaining in the cell suspension is smaller, but the proportion of fetal cells present is larger than was present in the starting sample.

The maternal blood sample can be a sample of whole blood or a fraction of whole blood (i.e., one resulting from treatment or processing of whole blood to increase the proportion of fetal nucleated cells present), referred to as a nucleated cell enriched sample. A nucleated cell enriched sample can be produced, for example, by separating non-nucleated, cells from nucleated cells within the maternal blood sample, resulting in a nucleated cell enriched sample. One method for separating non-nucleated cells from nucleated cells is by density gradient centrifugation, which separates cells on the basis of cell size and density. The maternal blood sample can be subjected to density gradient centrifugation using a density gradient material. Appropriate commercially available density gradient materials include Ficoll, Ficoll-Hypaque, Histopaque, Nycodenz and Polymorphprep. After centrifugation, the maternal blood sample can be separated into a supernatant layer, which contains platelets; a mononuclear cell layer; and an agglutinated pellet which contains non-nucleated erythrocytes. The mononuclear layer can be separated from the other layers, to produce a nucleated cell enriched sample from which non-nucleated cells have been removed and which is enriched in nucleated cells.

Another alternative to mononuclear cell isolation for production of a nucleated cell enriched sample is to selectively lyse maternal non-nucleated erythrocytes. Cells in the maternal blood sample can be incubated in one of a number of hypotonic buffers known to be effective, and conventionally used, for lysing nonnucleated erythrocytes, such as 0.17M NH₄Cl, 0.01M Tris, pH 7.3. Buffers suitable for this purpose are also available commercially (e.g. “Lyse and Fix”, GenTrak).

Internal cell markers can be used for detection. For example, fetal cell marker CD235a can be used. Detection of cells containing such markers can be accomplished using known techniques. For example, techniques for detection based on ZAP-70 can be adapted for the detection of fetal and other cell markers.

### D. Sorting and Separation of Fetal Cells

Following expansion and/or differentiation of fetal cells, the fetal cells can be separated using known techniques, such as flow cytometry, binding of cells to immunomagnetic beads or cell panning. In general, the monoclonal antibodies can be associated with a detectable label (e.g., radioactive material, fluorophore). This label may be conjugated directly to the monoclonal antibody with which the cells are contacted (the primary antibody) or it can be attached to a second antibody (a secondary antibody) which is specific for and recognizes the primary antibody, for example an anti-immunoglobulin constant region antibody. When cells are contacted with a combination of two or more primary antibodies, these antibodies can be labeled such that they can be distinguished from each other, e.g. each antibody can be labeled with a different fluorophore. A cell which is bound by multiple antibodies can then be identified by the presence of fluorescence from each of the different fluorophores associated with the cell.

Cells can be sorted and separated using any suitable means and technique. Many techniques for sorting cells are known and can be used with the disclosed methods. For example, cells can be sorted using microfluidic devices, polydimethylsiloxane (PDMS) devices, laser tweezers, optical switching, pressure switching, paramagnetic beads. Laser tweezers use the force of a focused laser beam to trap and move cells and particles (see, for example, Spalding and Dholakia, Nature 426, 421-424 (2003)). Optical switching uses the force of a laser beam to move a cell or particle form one flow stream to another (see, for example, Wang et al., Nature Biotech. 23(1):83-87 (2005)). Pressure switching can control liquid flow by manipulating external driving pressures (see, for example, PCT Application Publication No. WO/1997/045644). Paramagnetic beads can be used to sort cells by, for example, magnetic selection or magnetic activated cell sorting (see, for example, Miltenyi Biotec, www.miltenyibiotec.com)

In the case in which the cells are labeled with a fluorescent molecule, separation can be carried out by means of flow cytometry, in which fluorescently-labeled molecules are separated from unlabelled molecules. This results in separation of fetal cells from maternal cells. That this separation has occurred can be verified, using known techniques, such as microscopy and detection of fetal cell markers. Flow cytometry can be performed on fluorescently labeled cells using a fluorescent activated cell sorter (also called a flow cytometer). Cells treated with one or more fluorescently labeled antibodies are passed through a laser beam and fluorescent cells can be physically deflected into a test tube or onto a slide for collection. When a single fluorescently labeled antibody is used, labeled cells can be separated from unlabelled cells by sorting for each population. When two antibodies are used, each labeled with a different fluorophore, cells positive for one or the other fluorophore or positive for both fluorophores can be separated from unlabelled cells by sorting for each population. In addition to sorting cells based upon fluorescence, flow cytometry can further be used to characterize cells to permit identification of different cell types within a mixed cell population. When cells pass through the laser beam of a flow cytometer, laser light is scattered. This scattering can be converted to an electronic signal to produce a scatter profile. The scatter profile is a composite of two parameters: forward angle light scatter and side scatter. Forward angle light scatter is influenced by cell size whereas side scatter is influenced by cell granularity. Different cell types generate different, characteristic scatter profiles.

In addition to analyzing cells types based upon their scatter profile, it is possible to sort for particular cells types in a mixed cell population based upon their differing scatter profiles. Thus, cells in a cell population containing other cell types can be separated from other cell types based upon the characteristic scatter profile and therefore can be further enriched by sorting on this basis.

It is also possible to separate fetal cells from maternal cells by means other than flow cytometry. Such separation procedures may be used in conjunction with or independent of flow cytometry. Thus, other methods of fetal cell separation can be used. The separation method used can result in elimination of unwanted cells (“negative selection”) or isolation of rare but desirable cells (“positive selection”).

Separation of fetal cells can be achieved by use of immunomagnetic beads or by cell panning. The expanded fetal cells can be mixed with antibody-coated polymer particles containing magnetic cores. These immunomagnetic beads are commercially available coated with a variety of antibodies which can be used as a “primary antibody” for direct contact with cells of a maternal blood sample. Alternatively, immunomagnetic beads can be coated with a variety of antibodies which can be used as a “secondary antibody”, based upon their ability to recognize and bind to a primary antibody. For example, immunomagnetic beads coated with an antibody specific for mouse immunoglobulins can be used when the primary antibody is a mouse immunoglobulin. Immunomagnetic beads coated with a secondary antibody can either be preincubated with the primary antibody in the absence of cells to form a primary-secondary antibody complex which is capable of binding cells for which the primary antibody is specific or the primary antibody can be contacted with cells in solution and then the primary antibody-cell mixture can be contacted with the secondary antibody-coated immunomagnetic beads.

After contacting cells with an antibody-coated immunomagnetic bead, antibody-bound cells are isolated with, for example; a magnetic particle concentrator (e.g. a magnet). Fetal cells can be contacted with immunomagnetic beads which allow for binding of fetal cells and the fetal cells can be isolated by collecting cells which bind to these immunomagnetic beads (positive selection). For example, a mouse monoclonal antibody against CD86 can be preincubated with immunomagnetic beads coated with a monoclonal antibody specific for mouse immunoglobulins (e.g. an antibody which recognizes an appropriate mouse immunoglobulin constant region such as an IgG constant region) and these immunomagnetic beads are then contacted with the expanded fetal cells. Miltenyi Biotec supplies materials for magnetic separation and analysis of cells.

Internal cell markers can be used for separation and sorting. For example, fetal cell marker CD235a can be used. Separation and sorting of cells containing such markers can be accomplished using known techniques. For example, techniques for sorting based on ZAP-70 can be adapted for the sorting of fetal and other cell markers.

Other methods of separating fetal granulocytes from maternal cells can also be used, provided that they make it possible to differentiate between fetal cells and maternal cells, and to isolate one from the other.

Any suitable sorting or separating device, apparatus or instrument can be used to sort and separate cells and in the disclosed methods. Many such devices, apparatuses and instruments embodying useful techniques are known. For example, useful devices and techniques include flow sorters such as FACSAria from BD (www.bdbiossciences.com), EPICS ALTRA from Beckman Coulter (www.beckmancoulter.com) and MoFlo from DakoCytomation (www.dakousa.com); microfluidics based cell sorters such as those described in Wang et al., Nature Biotech. 23(1):83-87 (2005), Fu et al., Nature Biotech. 17:1109-1111 (1999), Fu et al., Anal. Chem. 74(11):2451-2457 (2002), Wolff et al., Lab Chip 3:22-27 (2003), and Macdonald et al., Nature 426:421-424 (2003); dielectrophoretic field-flow fractionation such as those described in Yang et al., Anal. Chem. 71:911-918 (1999), and Wang et al., Anal. Chem. 72(4):832-839 (2000); electrokinetic switched microfluidic sorter such as the one described in Dittrich and Schwille, Anal. Chem. 75(21):5767-5774 (2003); and the “spitter chip” from Caliper. The cited publications are hereby incorporated by reference in their entirety and in particular for their description of cell and particle sorting devices, apparatus and methods. Useful apparatus for sorting and separating cells, and in particular, for sorting and separating fetal cells from maternal blood also include apparatus such as those described in U.S. Pat. Nos. 6,778,724, 6,744,038, 6,815,664, 6,833,542, 6,936,811, and 7,068,874, and those described in U.S. Patent Application Publication Nos. 20030007894, 20050207940, and 20060060767, which are hereby incorporated by reference in their entirety and in particular for their description of cell and particle sorting devices, apparatus and methods. Microfluidic devices are particularly useful for handling small volumes and small numbers of cells as can be generated and manipulated in the disclosed methods.

For use in cell sorting in a microfluidic device, an optical switch can be triggered by detection of a fluorescence signal from target cells flowing in a microfluidic channel network upstream of the optical switch position. Other detection modalities such as light scattering can also be used for activation of the optical switch. The optical switch can be used to direct cells or particles into one of a multiple number of output channel flow streams without modifying the underlying flow. In this way, the desired cells are collected for further use. The flow in a microfluidic channel is typically laminar at a very low Reynolds number. Consequently, any cell flowing in a particular lamina, or flow stream, will stay in that flow stream in the absence of any forces transverse to the lamina. The optical switch utilizes optical forces on a cell to accomplish just this, the transport of cells transverse to the lamina to move the cells from a flow stream that exits a bifurcation junction through one output channel to a flow stream that exits the bifurcation junction through the second output channel. The force exerted on a particle by an optical beam is a function of the optical power and the relative optical properties of the particle and its surrounding fluid medium. Forces on the order of 1 pN/mW can be achieved for biological cells approximately 10 μm in diameter. While the optical force is small, the force necessary to deflect a cell into an adjacent flowstream is also small, e.g., 900 pN to move a 10 μm diameter cell, 20-40 μm laterally across the flow in a few milliseconds. This is the force necessary to overcome the viscous drag force on the cell at the velocity implied by this lateral motion.

### E. Analysis of Fetal Cells and Prenatal Diagnosis

The disclosed fetal cells are particularly useful for analyzing one or more characteristics of the fetal cells relevant to the heath, condition and prognosis of a gestating fetus. Any characteristic can be analyzed, such as genetic, physiological, chromosomal, genomic, proteomal, biochemical, and other cellular characteristics. Methods, techniques, assays and systems for such analysis are known and can be used with the disclosed fetal cells. The disclosed fetal cells can also be cultured, stored, differentiated, transformed, transfected, and used for testing, assays, production of biologicals, chemicals, and cellular components.

Detection and/or analysis of characteristics of fetal cells is a preferred use for the disclosed fetal cells. Thus, disclosed is a method of analyzing one or more of the fetal cells for one or more characteristics. The fetal cells can be fetal cells obtained, expanded and/or differentiated as described herein. The fetal cells can form colonies and one or more colonies of fetal cells can be harvested, where one or more of the expanded fetal CD34+ cells that are analyzed are derived from one or more of the harvested colonies. Analysis of fetal cells can involve prenatal diagnosis.

The characteristic(s) to be detected or analyzed can be any characteristic of the fetal cells. Numerous characteristics of cells are known, and any such characteristics can be analyzed in the disclosed fetal cells. For example, the characteristic can be genotype, phenotype, physiological function, biochemical function, or a combination. The characteristic can be the presence or absence of one or more particular nucleic acid sequences, or the presence or absence of particular mutations, alternative sequences, alleles, homologous sequence, and the like. The characteristic can be the sex of the fetus from which the fetal cells derived. The sex of the fetus can be analyzed, for example, by detecting the presence of Y chromosomes, X chromosomes, or both in the fetal cells.

The characteristic can be a disease or condition or an indicator of a disease or condition. The indicator of the disease or condition can be analyzed by detecting one or more mutations, single nucleotide polymorphisms, genetic markers, or a combination associated with the disease or condition. The mutation, single nucleotide polymorphism, or genetic marker can be, for example, a cystic fibrosis-associated mutation, single nucleotide polymorphism, or genetic marker, a Duchenne muscular dystrophy-associated mutation, single nucleotide polymorphism, or genetic marker, a hemophilia A-associated mutation, single nucleotide polymorphism, or genetic marker, a Gaucher disease-associated mutation, single nucleotide polymorphism, or genetic marker, a sickle cell anemia-associated mutation, single nucleotide polymorphism, or genetic marker, a Tay-Sachs-associated mutation, single nucleotide polymorphism; or genetic marker, or a combination.

The characteristic can be a chromosomal abnormality. The chromosomal abnormality can be chromosomal aneuploidy, chromosomal translocation, deletion, duplication or a combination. The chromosomal aneuploidy can be trisomy 21, trisomy 18, trisomy 13 or a combination.

Numerous tests, assays, and techniques are known for detecting or analyzing cell characteristics, and such tests, assays and techniques can be used to analyze the disclosed fetal cells.

The term “prenatal diagnosis” means both the identification of a particular characteristic of the fetus (for example the sex) or the identification of a genetic anomaly or any type of genetic pathology (DNA alteration), infectious disease (viral, bacterial or parasitic) or metabolic disease (alteration to the synthesis of messenger RNA and/or proteins) which can be detected from a genetic analysis of isolated fetal cells. Thus, depending on the selected implementations of the disclosed method, prenatal diagnosis can be, for example, identifying a genetic anomaly or chromosomal anomaly on the DNA of a fetal cell, a genetic or infectious disease (viral, bacterial or parasitic) or identifying a precise genotype; in particular the genetic sex of the fetus. The term “slightly invasive or non-invasive method” means a method that does not involve the removal of tissues or fetal cells by biopsy and/or effraction from the placentary barrier.

The disclosed prenatal diagnosis involves fetal cells obtained using the disclosed methods. Such fetal cells can be obtained from a blood sample from a pregnant subject. In some forms of the method, a sample of maternal blood is removed early on in pregnancy (for example at about the fifth week of a human pregnancy). However, removing the sample of maternal blood and the expansion of fetal cells can be carried out at any time from the start to the end of pregnancy. For example, sampling and expansion of fetal cells can be carried out between the 7th and 15th week of pregnancy or between the 10th and 15th week of pregnancy.

When identifying a chromosomal anomaly or the sex of a fetus, in situ hybridization of probes specific to the chromosomal anomaly or the sex to be detected can be used. Specific probes for a chromosomal sequence can be DNA or PNA (peptide nucleic acid) type probes (Lohse et al., PNAS 1999 96: 11804-11808). One example of an in situ hybridization technique is known as FISH (Fluorescence In Situ Hybridization) (Poon et al., Clin Chem 2000; 46(11):1832-4), but any method that is known to the skilled person that can detect a chromosomal anomaly or sex chromosomes on the genome of a cell using specific probes can be used in the context of the invention.

The term “genetic target” means any genetic characteristic, for example a particular mutation of a gene, specifically associated with a phenotype or a genetic disease or infectious disease of the fetus. The term “polymorphism marker” means any characteristic that can be identified in DNA the presence of which is correlated with a particular genotype. These markers can distinguish paternal DNA from maternal DNA and thus can demonstrate the bi-parental composition of fetal DNA. Examples of markers that can be cited are restriction fragment length polymorphism (RFLP) markers, SNP (Single Nucleotide Polymorphism) markers, microsatellite markers, VNTR (Variable Number of Tandem Repeats) markers or STR (Short Tandem Repeats) markers.

Microsatellite markers are particularly useful for the characterization of cells and for implementing prenatal diagnosis. In some forms of the method, at least one marker for polymorphism to be identified can be a microsatellite marker, a VNTR (Variable Number of Tandem Repeats) marker or a STR (Short Tandem Repeats) marker. These have the advantage of being identifiable by amplification using specific primers. Microsatellite markers, VNTR or STR, are composed of tandem repeats, usually polyCA/GT moieties. Allelic variations, due to a variation in the number of repeats, are readily detected by PCR type amplification using primers corresponding to the unique sequences flanking the microsatellite. Using this methodology, the presence of particular microsatellite markers can be specifically researched, in particular as a genetic target, for prenatal diagnosis, in particular for the diagnosis of particular chromosomal changes.

Prenatal diagnosis can be used in particular when seeking a genetic or chromosomal anomaly of the fetus or a particular genotype thereof by hybridizing all or a portion of the preamplified DNA preparation using specific DNA probes. The DNA probes can be selected so that they hybridize specifically to genetic targets or polymorphisms for their identification, or to sequences carrying the genetic target(s) to be identified. Hybridization of the probes to the genetic targets can be detected using conventional techniques for detecting hybridization complexes of nucleic acids of the slot blot, Southern blot or advantageously now using DNA micro- or macro-arrays. Molecular probes can, for example, be selected for the specific detection of cystic fibrosis, muscular dystrophies, Gaucher's disease, haemoglobinopathies, haemophilia, penylketonurias and cystic fibrosis.

In some forms of the method, DNA probes specific for genetic targets to be identified can be fixed to a support forming a DNA micro- or macro-array. The preamplified DNA preparation can be, for example, labeled with a radioactive or fluorescent marker and brought into contact with the DNA micro- or macro-array including the specific probes. The hybridization intensity can be measured for each spot containing a specific probe, thus providing great sensitivity of determination of the presence of the desired markers on the DNA of a collected cell.

An alternative method for determining chromosomal anomalies and in particular gains and losses of chromosomes for prenatal diagnosis is the comparative genomic hybridization method (CGH) consisting (i) of comparing hybridization on a chromosomal or cosmid preparation or on a DNA array, preparing pre-amplified DNA derived from the genome of a single fetal cell, and preparing pre-amplified DNA from cells of maternal origin or non-fetal reference cells, the two preparations having been labeled with different markers, and (ii) identifying differences in hybridization between the DNA of the collected cell after filtration and maternal DNA (Voullaire et al., Prenat Diagn 1999 19: 846-851). In one implementation of the invention, prenatal diagnosis can be carried out by means of comparative genomic hybridization (CGH) of a preamplified DNA preparation derived from the DNA of a single fetal cell, and of a preamplified DNA preparation of cells of maternal origin or of non-fetal reference cells.

Where the presence of a selected nucleic acid of interest in fetal nucleic acid is to be determined (detected and/or quantitated), the isolated fetal cells can be treated to render nucleic acid present in them available for amplification. Amplification of fetal nucleic acid, e.g. DNA, from fetal cells can be carried out using a known amplification techniques, such as the polymerase chain reaction (PCR). Amplified fetal DNA can be subsequently separated on the basis of size (e.g. by gel electrophoresis) and contacted with a selected labeled probe, such as labeled nucleic acid complementary to a nucleic acid of interest (e.g. complementary to an abnormal gene or gene portion, or Y-specific DNA). Detection of the labeled probe after it has hybridized to fetal DNA results in detection of the sequence of interest in the fetal DNA. Quantitation of the hybridized labeled probe results in quantitation of the fetal DNA.

Chromosomal abnormalities in a fetus can be analyzed using the fetal cells. For example, fetal cells isolated as disclosed can be separated onto a solid support, such as a slide, and screened for chromosomal abnormalities using in situ hybridization. In this form of the method, a selected nucleic acid probe, such as a labeled DNA probe for chromosomal DNA associated with a chromosomal abnormality, can be combined with fetal DNA under conditions appropriate for hybridization of complementary sequences to occur. Detection and/or quantitation of the labeled probe after hybridization results in detection and/or quantitation of the fetal DNA to which the probe has hybridized. A difference or differences in the hybridization of the labeled DNA probe to fetal DNA as compared to hybridization of the labeled DNA probe to DNA from a normal cell (i.e. a cell which does not have a chromosomal abnormality in the DNA of interest) can be detected as an indication of the presence of a chromosomal abnormality in the fetal DNA. For example, a trisomy in the fetal DNA can be detected by hybridization of a labeled DNA probe to three chromosomes in the fetal DNA as compared to hybridization to only two chromosomes in normal cells.

The sex of a fetus can be determined by analyzing the fetal cells. For example, cells isolated as disclosed can be separated onto a solid support and screened for presence or absence of Y chromosomal DNA by in situ hybridization using a nucleic acid probe which is specific for the Y chromosome. Presence of hybridization of the Y chromosome-specific probe is indicative of a male fetus whereas absence of hybridization is indicative of a female fetus.

Following expansion and/or differentiation of fetal cells, the fetal cells can be used as a source of fetal nucleic acid for analyses such as determination of fetal gender, detection of a genetic disease in the fetus or detection of a chromosomal abnormality in the fetus. Fetal nucleic acid in fetal cells can be analyzed or assessed for the occurrence of a nucleic acid of interest for diagnostic or other purposes. The nucleic acid which is to be detected in fetal cells is referred to herein as a nucleic acid of interest. For example, the nucleic acid of interest whose presence or absence is to be determined and whose quantity can also be determined may be a gene for a disease, such as cystic fibrosis, where the causative gene or gene portion has been cloned and sequenced; alternatively, the nucleic acid of interest may be X- or Y-chromosome-specific DNA. The same procedure can also be used, with appropriate modifications (e.g., an appropriate nucleic acid probe, time, temperature), to detect other genes or gene portions. The nucleic acid detected in fetal cells, that is, the nucleic acid of interest, can be DNA, e.g. chromosomal DNA or a particular gene fragment within chromosomal DNA or amplified from chromosomal DNA, or can be RNA, e.g. mRNA. The labeled probe used to detect the nucleic acid of interest can be, for example, a labeled DNA probe, a labeled RNA probe or labeled oligonucleotides.

Fetal cells can be treated such that fetal nucleic acid is made available for detection. Appropriate treatments that can be used, depending on the method used for detection of fetal nucleic acid. For example, fetal DNA can be made available by boiling the fetal cells to lyse them, thereby releasing fetal DNA, for instance prior to amplification of fetal DNA. Fetal granulocytes can be attached to a solid support, e.g. a microscope slide, in such a way that fetal nucleic acid is made available, for example by fixing fetal cells or nuclei to a microscope slide prior to in situ hybridization. The fetal cells or portions thereof (e.g. nuclei) which are attached to a solid support such that fetal nucleic acid is made available is referred to as fetal granulocyte material. Fetal nucleic acid in fetal cells (or fetal cell material) can be detected directly, for example by in situ hybridization of a labeled nucleic acid probe complementary to a nucleic acid of interest or the fetal nucleic acid can be amplified prior to detection using a known amplification technique such as the polymerase chain reaction (PCR). Primers for PCR amplification can be chosen which specifically amplify a DNA of interest in the fetal DNA.

If in situ hybridization is to be carried out, fetal cells can be separated onto a solid support, such as a microscope slide, such that fetal nucleic acid is available for detection. In situ hybridization can be used, for example, to detect Y chromosome-specific sequences in fetal DNA in order to determine the gender of a fetus. In situ hybridization can also be used to assess chromosomal abnormalities in a fetus, including chromosomal aneuploidies, such as a trisomy, or chromosomal rearrangements or deletions.

Fetal DNA can be amplified by PCR. If amplification is to be carried out, fetal cells can be lysed by boiling and fetal DNA can then be amplified for an appropriate number of cycles of denaturation and annealing (e.g., approximately 24-60). Control samples include a tube without added DNA to monitor for false positive amplification. More than one separate fetal gene can be amplified simultaneously (multiplex detection). When amplification is carried out, the resulting amplification product is a mixture which contains amplified fetal DNA of interest (i.e., the DNA whose presence is to be detected and/or quantitated) and other DNA sequences. Subsequent analysis of amplified DNA can be carried out using known techniques, such as: digestion with a restriction endonuclease, ultraviolet light visualization of ethidium bromide stained agarose gels, DNA sequencing, or hybridization with a labeled DNA probe, for example, allele specific oligonucleotide probes, or hybridized to nucleic acid arrays. Thus, the method can be used for all nucleic acid-based diagnostic procedures currently being achieved with other methods, such as amniocentesis.

The presence of fetal nucleic acid associated with diseases or conditions can be detected and/or quantitated by the present method. In each case, an appropriate probe is used to detect the sequence of interest. For example, for prenatal detection of cystic fibrosis, a labeled DNA probe complementary to the gene associated with cystic fibrosis can be used. A suitable probe is described in Newton, C. R., et al. Lancet 2, 1481-1483 (1989). Sequences from probes St14 (Oberle, I., et al., New Engl. J. Med., 312, 682-686 (1985)), 49a (Guerin, P., et al., Nucleic Acids Res., 16, 7759 (1988)), KM-19 (Gasparini, P., et al., Prenat. Diagnosis, 9, 349-355 (1989)), or the deletion-prone exons for the Duchenne muscular dystrophy (DMD) gene (Chamberlain, J. S., et al., Nucleic Acids Res., 16, 11141-11156 (1988)) are used as probes. St14 is a highly polymorphic sequence isolated from the long arm of the X chromosome that has potential usefulness in distinguishing female DNA from maternal DNA. It maps near the gene for Factor VIII:C and, thus can also be utilized for prenatal diagnosis of Hemophilia A. Primers corresponding to sequences flanking the six most commonly deleted exons in the DMD gene, which have been successfully used to diagnose DMD by PCR, can also be used (Chamberlain et al., Nucleic Acids Res., 16, 11141-11156(1988)). Other conditions which can be diagnosed by the present method include β-thalassemia (Cai et. al., Blood, 73:372-374 (1989); Cai et al., Am. J. Hum. Genet., 45:112-114 (1989); Saiki, R. K., et al., New Engl. J. Med., 319, 537-541 (1988)), sickle cell anemia (Saiki et al., New Engl. J. Med., 319, 537-541 (1988)), phenylketonuria (DiLella et al., Lancet, 1,497-499 (1988)) and Gaucher disease (Theophilus et al., Am. J. Hum. Genet., 45, 212-215 (1989)). An appropriate probe (or probes) is available for use in the present method for assessing each condition.

### Examples

### F. Example 1

### Expansion of Fetal Cells Using Various Factors

Fetal and adult cells were grown in the presence of different factors to assess their effect on differential expansion of fetal cells. The factors were Stem Cell Factor at 50 ng/mL, IL-3 at 5 ng/mL, IL-6 at 5 ng/mL, EPO at 1.5 U/mL, TPO at 100 ng/mL, and Flt-3 at 50 ng/mL. The cells were CD34+ positive cells purified from adult mobilized donor peripheral blood and CD34+ positive cells purified from fetal liver tissue purchased from Cambrex (Walkersville, Md.). Cells were plated at 10,000 cells per ml into 24-well tissue culture plates. Cells were incubated in HPGM medium with 50 units/ml of penicillin, 50 μg/ml streptomycin sulfate and the cytokine combinations above for 6 days at 37° C. and 5% CO₂ in a humidified chamber. After 6 days, an aliquot of cells was counted manually with a hemacytometer and using a standard formula, the total cell numbers were calculated. An additional aliquot was used to assay total ATP levels (linear correlation with total cell numbers) using a ViaLight assay kit (Cambrex, Walkersville, Md.).

Table 1 shows initial and final cell counts for fetal and adult cells when cultured with various combinations of the factors (all including SCF). The fourth column in shows the fold expansion of fetal cells for some growth conditions. In every case measured, the fetal cells expanded more than adult cells (compare fourth column to the seventh column) The final column shows the ratio of fetal cells to adult cells for some grow conditions. In all cases where the ratio was measured, fetal cells were more numerous after expansion.

TABLE 1

| | Total | Fetal | Fold | Total | Adult | Fold | Differential | Differential |
|---|---|---|---|---|---|---|---|---|
| Cytokine | ATP/well | cells | expansion | ATP/well | cells | expansion | ATP | count |
| Combination | (AU) | per well | (fetal) | (AU) | per well | (adult) | Fetal/Adult | Fetal/Adult |
| O | 77.2 | 188500 | 18.85 | 0.8 | 7500 | 0.75 | 93.6 | 25.1 |
| OA | 272.8 | 577500 | 57.75 | 46.2 | 91200 | 9.12 | 5.9 | 6.3 |
| OB | 167.9 | 410400 | 41.04 | 8.9 | 23400 | 2.34 | 18.8 | 17.5 |
| OC | 657.9 | 1410000 | 141 | 100.7 | 127500 | 12.75 | 6.5 | 11.1 |
| OD | 267.0 | 652500 | 65.25 | 18.3 | 37000 | 3.7 | 14.6 | 17.6 |
| OE | 213.5 | 663300 | 66.33 | 70.9 | 150000 | 15 | 3.0 | 4.4 |
| OAB | 280.2 | | | 57.5 | | | 4.9 | |
| OAC | 668.3 | | | 282.6 | | | 2.4 | |
| AOD | 457.9 | | | 95.0 | | | 4.8 | |
| OAE | 517.1 | | | 186.0 | | | 2.8 | |
| OBC | 741.4 | | | 225.8 | | | 3.3 | |
| OBD | 335.9 | 850500 | 85.05 | 38.7 | 123200 | 12.32 | 8.7 | 6.9 |
| OBE | 341.2 | | | 138.6 | | | 2.5 | |
| OCD | 706.0 | | | 260.2 | | | 2.7 | |
| OCE | 638.6 | | | 338.1 | | | 1.9 | |
| ODE | 480.5 | | | 157.6 | | | 3.0 | |
| OABC | 691.0 | | | 323.9 | | | 2.1 | |
| OABD | 389.8 | | | 92.8 | | | 4.2 | |
| OABE | 281.3 | | | 126.5 | | | 2.2 | |
| OACD | 689.6 | | | 326.3 | | | 2.1 | |
| OACE | 609.6 | | | 347.3 | | | 1.8 | |
| OADE | 508.0 | | | 181.6 | | | 2.8 | |
| OBCD | 694.0 | | | 268.7 | | | 2.6 | |
| OBCE | 543.4 | | | 228.4 | | | 2.4 | |
| OBDE | 299.0 | | | 122.2 | | | 2.4 | |
| OCDE | 670.0 | | | 283.4 | | | 2.4 | |
| OABCD | 568.6 | | | 259.4 | | | 2.2 | |
| OABCE | 602.1 | | | 289.0 | | | 2.1 | |
| OABDE | 317.4 | | | 137.4 | | | 2.3 | |
| OACDE | 472.0 | | | 165.0 | | | 2.9 | |
| OBCDE | 441.0 | 1449000 | 144.9 | 215.1 | 292000 | 29.2 | 2.1 | 5.0 |
| OABCDE | 571.3 | 1577000 | 157.7 | 259.8 | 440800 | 44.08 | 2.2 | 3.6 |
| OC | | 1000000 | 100 | | 220000 | 22 | | 4.5 |
| OCD | | 1200000 | 120 | | 350000 | 35 | | 3.4 |
| O | | 450000 | 45 | | 30000 | 3 | | 15.0 |
| OA | | 75000 | 7.5 | | 50000 | 5 | | 1.5 |
| OB | | 550000 | 55 | | 30000 | 3 | | 18.3 |
| OAB | | 625000 | 62.5 | | 100000 | 10 | | 6.3 |
| OE | | 900000 | 90 | | 130000 | 13 | | 6.9 |
| ODE | | 1250000 | 125 | | 160000 | 16 | | 7.8 |

O Stem Cell Factor 50 ng/mL
A IL-3 5 ng/mL
B IL-6 5 ng/mL
C EPO 1.5 U/mL
D TPO 100 ng/mL
E Flt-3 50 ng/mL

Expansion of fetal cells was further assessed using different concentrations of three factors: SCF, LI-3, and IL-6. Tables 2 and 3 show adult and fetal cell counts (Table 2) and ratios of adult and fetal cells (Table 3) following expansion. In all cases (except in the absence of any of the factors), fetal cells expanded more than adult cells. Differential expansion was significant and greater in the presence of SCF. Based on these results, differential expansion of fetal cells can be best accomplished by incubation in the presence of SCF, with a concentration of 50 ng/mL or more being preferred. IL-3 and IL-6 also aid differential expansion of fetal cells by SCF, with incubation in the presence of 10 ng/mL of more of IL-3 and 5 ng/mL or more of IL-6 being preferred. Expansion for 8 days provided greater differential expansion than expansion for 6 days.

TABLE 2

| | ADULT | | | | FETAL | | |
|---|---|---|---|---|---|---|---|
| | cells/well (millions) | | | | cells/well (millions) | | |
| IL-6 = 0 | IL3 | | | IL-6 = 0 | IL3 | | |
| SCF | 0 | 5 | 10 | SCF | 0 | 5 | 10 |
| 0 | 0.05 | 0.05 | 0.05 | 0 | 0.05 | 0.075 | 0.125 |
| 25 | 0.05 | 0.05 | 0.05 | 25 | 0.35 | 0.6 | 0.625 |
| 50 | 0.05 | 0.1 | 0.1 | 50 | 0.45 | 0.675 | 0.6 |
| 100 | 0.05 | 0.1 | 0.1 | 100 | 0.45 | 0.675 | 0.7 |
| | ADULT | | | | FETAL | | |
| | cells/well (millions) | | | | cells/well (millions) | | |
| IL-6 = 5 | IL3 | | | IL-6 = 5 | IL3 | | |
| SCF | 0 | 5 | 10 | SCF | 0 | 5 | 10 |
| 0 | 0.05 | 0.05 | 0.05 | 0 | 0.075 | 0.1 | 0.1 |
| 25 | 0.05 | 0.05 | 0.05 | 25 | 0.45 | 0.6 | 0.75 |
| 50 | 0.05 | 0.1 | 0.05 | 50 | 0.525 | 0.675 | 0.75 |
| 100 | 0.05 | 0.1 | 0.05 | 100 | 0.55 | 0.625 | 0.725 |
| | ADULT | | | | FETAL | | |
| | cells/well (millions) | | | | cells/well (millions) | | |
| IL-6 = 10 | IL3 | | | IL-6 = 10 | IL3 | | |
| SCF | 0 | 5 | 10 | SCF | 0 | 5 | 10 |
| 0 | 0.05 | 0.05 | 0.05 | 0 | 0.05 | 0.1 | 0.1 |
| 25 | 0.05 | 0.05 | 0.05 | 25 | 0.525 | 0.675 | 0.675 |
| 50 | 0.05 | 0.1 | 0.05 | 50 | 0.6 | 0.7 | 0.675 |
| 100 | 0.05 | 0.1 | 0.05 | 100 | 0.6 | 0.775 | 0.75 |

TABLE 3
IL-6 = 0

| | Ratio | | |
|---|---|---|---|
| | IL3 | | |
| SCF | 0 | 5 | 10 |
| 0 | 1 | 1.5 | 2.5 |
| 25 | 7 | 12 | 12.5 |
| 50 | 9 | 6.75 | 6 |
| 100 | 9 | 6.75 | 7 |

IL-6 = 5

| | Ratio | | |
|---|---|---|---|
| | IL3 | | |
| SCF | 0 | 5 | 10 |
| 0 | 1.5 | 2 | 2 |
| 25 | 9 | 12 | 15 |
| 50 | 10.5 | 6.75 | 15 |
| 100 | 11 | 6.25 | 14.5 |

IL-6 = 10

| | Ratio | | |
|---|---|---|---|
| | IL3 | | |
| SCF | 0 | 5 | 10 |
| 0 | 1 | 2 | 2 |
| 25 | 10.5 | 13.5 | 13.5 |
| 50 | 12 | 7 | 13.5 |
| 100 | 12 | 7.75 | 15 |

### G. Example 2

### Differential Expansion of Fetal Cells in Spiked Cell Sample

Examples of the disclosed method for expansion of fetal cells were carried out using blood collected from women not believed to be pregnant that was spiked with the addition of male fetal liver CD34+ cells. Five different protocols were used to assess various factors. All protocols used drawn female blood, red blood cell lysis, enrichment of CD34+ cells, and culturing under fetal cell differential expansion conditions. Cells were cultured in HPGM with 100 ng/ml SCF. Cells were counted by hemacytometer and male cells were detected using a fluorescent in situ hybridization (FISH) assay for X and Y chromosome detection (XY-FISH) at various points during the protocols. Samples of whole blood, RBC lysed blood (total white blood cells) column flow through, pooled washes, and enriched cell populations prior to and following 6 days of culture were tested for the presence of nuclei.

For FISH assays, cells were incubated in 0.075 M KCL for 18 minutes at 37° C. Cells were fixed, dehydrated and the cytoplasm removed by additions of ice-cold Carnoy's fixative (MeOH:glacial acetic acid, 3:1). Cells were adhered to glass slides by air drying. Dual fluorescent-labeled probes for specific regions of the X and Y chromosomes were added (Aquarius Probes Chromosome X Alpha and Y Classical Satellite Probes, Cambridge, UK). DNA was denatured for 90 seconds at 75° C. and allowed to re-anneal overnight at 37° C. If the specific chromosome is present, the fluorescent probes can hybridize. Non-specific binding was removed by two washes of increasing stringency. DAPI was added to allow the nuclei to be visualized. Nuclei were examined at 100× in oil immersion to observe the nuclei and note the presence of two X chromosome probes or of one X and one Y chromosome probe.

In the first test, 12 ml of blood were drawn, the blood was subjected to red blood cell lysis, and CD34+ cells were enriched using magnetic beads. At this point there were 125,000 adult cells present. XY-FISH showed no males cells (the expected result). 12,500 fetal cells were then added to the adult cells (1:10 ratio). XY-FISH showed males cells as expected. The spiked cell mixture was plated at 10,000 cells per well (about 9,000 adult and 1,000 fetal cells per well) and cultured for 5 days. After culture there were 20,000 to 40,000 cells per well. XY-FISH showed that 90% of the cells were males cells. Fetal cell expanded 20-40 fold while adult cells did not expand.

In the second test, 12 ml of blood were drawn and 100,000 fetal cells were added. The spiked blood was subjected to red blood cell lysis. Approximately 30 million cells were present. XY-FISH showed males cells as expected. CD34+ cells were then enriched using magnetic beads. At this point there were 200,000 cells present. XY-FISH showed no males cells in the flow-through and approximately 10% male cells in the CD34+ fraction. The spiked cell mixture was plated at 10,000 cells per well and cultured for 6 days. XY-FISH showed males cells as expected, but the cells were not counted. More male cells were observed than in the third test as expected.

In the third test, 12 ml of blood were drawn and 10,000 fetal cells were added. The spiked blood was subjected to red blood cell lysis. Approximately 30 million cells were present. XY-FISH showed males cells as expected. CD34+ cells were then enriched using magnetic beads. At this point there were 130,000 cells present. XY-FISH showed no males cells in the flow-through and approximately 5% male cells in the CD34+ fraction. The spiked cell mixture was plated at 10,000 cells per well and cultured for 6 days. XY-FISH showed males cells as expected, but the cells were not counted. Fewer male cells were observed than in the second test as expected.

In the fourth test, 10 ml of blood were drawn and 100 fetal cells were added. The spiked blood was subjected to red blood cell lysis. Approximately 30 million cells were present. CD34+ cells were then enriched using magnetic beads. At this point there were 50,000 to 100,000 cells present. The spiked cell mixture was plated in a single well and cultured for 6 days. XY-FISH showed no males cells and very few total cells. Less than 0.5% of the cells survived the culture step.

In the fifth test, 10 ml of blood were drawn and 100 fetal cells were added. The spiked blood was subjected to red blood cell lysis. Approximately 30 million cells were present. CD34+ cells were not enriched. The spiked cell mixture was plated in two wells and cultured for 6 days. XY-FISH showed a few males cells. It was not clear if the fetal cells had expanded.

These tests showed that fetal cells could survive processing in the method and be expanded.

### H. Example 3

### Differential Expansion of Fetal Cells in Spiked Cell Sample

Examples of the disclosed method for expansion of fetal cells were carried out using blood collected from seven women 12-17 weeks pregnant. Two different protocols were used to assess the effect of sample size and CD34+ enrichment. All protocols used drawn female blood, red blood cell lysis, and culturing under fetal cell differential expansion conditions. Cells were incubated in HPGM medium with 50 units/ml of penicillin, 50 μg/ml streptomycin sulfate and 100 ng/ml SCF for 6 days at 37° C. and 5% CO₂ in a humidified chamber. Male cells were detected using a FISH assay for X and Y chromosome detection (XY-FISH). FISH assays were performed as described in Example 2.

RBC lysis was performed by incubating the whole blood in 16 volumes of hemolytic lysis buffer at 37° C. for 5 minutes. Hemolytic lysis buffer consists of 8.26 grams of ammonium chloride, 1.0 gram potassium bicarbonate and 0.32 grams EDTA tertrasodium per liter of deionized water, pH 7.0-7.4. After cetrifugation at 400×g for 10 minutes, the supernatant was removed and the white blood cell pellet was suspended in 2-3 ml of autologous plasma. 100 μl of CD34 magnetic beads and 100 μl of FC blocking reagent (CD34 multisort kit, human, Miltenyi, Auburn, Calif.) were added and the cells incubated for 30 minutes at 4° C. After incubation, the cell suspension was washed with 15 ml of Auto-MACS buffer (Miltenyi, Auburn, Calif.). and the cell pellet was resuspended in 2-3 ml of the same buffer. The cell suspension was applied to a LS column in a magnetic field (Miltenyi, Auburn, Calif.) and allowed to flow by gravity feed. Five 1 ml washes of Auto-NACS buffer were gravity flowed through the column. The column was the removed from the magnetic field and the target cells dislodged from the column in 1 ml of Auto-MACS buffer using a plunger.

In the first test, 30 ml of blood were drawn and subjected to red blood cell lysis. CD34+ cells were then enriched using magnetic beads. The cells were plated and cultured for 6 days. FISH assays were preformed as described in Example 2. Male nuclei were identified in three samples. These three samples were confirmed by the physician to be from patients carrying male fetuses (as determined by ultrasound). In four samples, only female nuclei were identified. Three of these samples were confirmed by the physician to be from patients carrying female fetuses (by ultrasound). The fourth sample came from a patient confirmed to be carrying one male and one female fetus (by ultrasound).

In the second test, 10 ml of blood were drawn and subjected to red blood cell lysis. CD34+ cells were not enriched. The cells were plated and cultured for 6 days. As in the first test, male nuclei were identified in three samples. These three samples were confirmed by the physician to be from patients carrying male fetuses (as determined by ultrasound). In four samples, only female nuclei were identified. Three of these samples were confirmed by the physician to be from patients carrying female fetuses (by ultrasound). The fourth sample came from a patient confirmed to be carrying one male and one female fetus (by ultrasound).

### I. Example 4

### Differential Expansion of Fetal Cell Colonies

CD34+ cells purified from adult mobilized donor peripheral blood and CD34+ cells purified from fetal liver tissue were purchased from Cambrex (Walkersville, Md.). The cells were plated at 1000-5000 cells per 3 cm² tissue culture dish in semi-solid media (methylcellulose) and incubated in HPGM medium with 100 ng/ml SCF and with or without IL-3 and IL-6. Normal (non-pregnant) female blood was collected, debulked as described in Example 3 and enriched for CD34+ cells. The enriched cells were plated at 1000-5000 cells per 3 cm² tissue culture dish in semi-solid media (methylcellulose) and incubated in HPGM medium with 100 ng/ml SCF. Colony formation was monitored after 6-12 days of incubation. No colony formation was observed for adult CD34+ cells while fetal cells did form colonies. Microscopically visible colonies of fetal cells formed at approximately 5-6 days and continued to expand for up to 12 days.

### J. Example 5

### Fetal Cell Expansion in Patient Samples as Determined by QPCR

Examples of the disclosed method for expansion of fetal cells were carried out with cells enriched from pregnant maternal blood following elective termination. Two different enrichment methods were used prior to culture of the resulting cells. Following cell enrichment, samples were divided for QPCR analysis both prior to and following culture in HPGM media containing Stem Cell Factor. Both protocols used drawn female blood with a known male pregnancy, and enrichment for cells of hematopoietic precursor status. Two samples had observed fetal cell growth by quantitating with PCR the presence of male cells prior to and following eight days of growth.

| | | | Minimum |
|---|---|---|---|
| | Male/Fetal | Male/Fetal | detected |
| Sample | Cells - Day 0 | Cells - Day 8 | expansion |
| A | 82 | 238 | 2.9 |
| B | 0 | 188 | 188.0 |

In the sample A, 43 ml of maternal blood were drawn from a 21 week gestation pregnancy, the blood was subjected to red blood cell lysis and Ficoll gradient purification, yielding 6×10⁷ cells. The PBMCs were processed with the Miltenyi lineage depletion MACS protocol, yielding 179,000 cells. The lineage committed cells were frozen and the progenitor cells were counted and divided into two portions. One hundred thousand cells were plated in HPGM with 100 ng/ml of SCF for 8 days, and 50,000 cells were subjected to DNA extraction using a commercially available genomic DNA extraction kit. The extracted DNA was used in real-time quantitative PCR using Taqman probes to detect both GAPDH for total DNA and DYS14 as a Y-chromosome specific quantitation. Following culture, the 33,000 cells that remained were subjected to the same DNA extraction and QPCR procedure. The ratio of DYS14 to GAPDH was used to determine percent male cells (fetal cells) present in the maternal blood. This percentage what then applied to the total number of cells present both pre- and post-culture to determine the total number of male cells at each stage. This indicated an expansion of nearly 3 fold following culture with an increase in fetal percentage from 0.05% to 0.8% of the total cells present.

For sample B, 27 ml of maternal blood were drawn from a 13 week gestation pregnancy, and the blood was subjected to StemCell company Human Progenitor Enrichment Cocktail RosetteSep protocol, yielding 168,000 cells. The progenitor cells were counted and divided equally into two portions. Approximately 80,000 cells were plated in HPGM with 100 ng/ml of SCF for 8 days, and 80,000 cells were subjected to DNA extraction using a commercially available genomic DNA extraction kit. The extracted DNA was used in real-time quantitative PCR using Taqman probes to detect both GAPDH for total DNA and DYS14 as a Y-chromosome specific quantitation. Following culture, the 25,000 cells that remained were subjected to the same DNA extraction and QPCR procedure. The ratio of DYS 14 to GAPDH was used to determine percent male cells (fetal cells) present in the maternal blood. This percentage what then applied to the total number of cells present both pre- and post-culture to determine the total number of male cells at each stage. Prior to culture, there were too few male cells present to accurately quantitate above background levels, resulting in a quantitation of 0 cells, however growth indicates that at least one cell was present per 80,000 cells. Following culture 188 cells were calculated, indicating an expansion of up to 188 fold.

### K. Example 6

### Fetal Cell Expansion in Patient Samples as Determined by Limited Dilution Cloning and QPCR

An additional example of the disclosed method for expansion of fetal cells was again carried out with cells enriched from pregnant maternal blood following elective termination. Following cell enrichment, the sample was diluted to 10 cells per well and cultured in HPGM media containing Stem Cell Factor. The sample had observed fetal cell growth by directly counting cells in the wells, and the identification of fetal was performed with PCR for the presence of the Y-chromosome.

For this dilution cloning sample, 32 ml of maternal blood were drawn from an 18 week gestation pregnancy following elective termination. The blood was subjected to red blood cell lysis and Ficoll gradient purification, yielding 1.2×10⁷ PBMCs. These cells were subjected to Miltenyi MACS lineage depletion, and the resulting 132,000 cells were incubated overnight in HPGM with 100 ng/ml of SCF. From the overnight plating, 23,000 live cells were plated in multiple 384-well plates at approximately 10 live cells per well. These cells were cultured for 10 days, and then visually inspected by microscope. Over 90% of all wells were empty, while the remaining 10% had 10 cells or less. A total of seven wells with greater than 30 cells were harvested for analysis by QPCR. One well had over 500 cells, one had approximately 100 cells, and the remaining five wells had approximately 30 cells. These cells were subjected to DNA extraction and PCR of these cells with GAPDH and DYS14 for Y-chromosome. It was determined that the expanded cells were male, and therefore fetal in origin. The range of expansion found from these positive wells was 3-fold to 50-fold. This example demonstrates the significant differential growth of fetal cells relative to maternal cells using the disclosed method.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a fetal cell” includes a plurality of such fetal cells, reference to “the fetal” is a reference to one or more fetal cells and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives; components, integers or steps.

Those skilled in the art will recognize, or be able to ascertain using no more than routine experimentation, many equivalents to the specific embodiments of the method and compositions described herein. Such equivalents are intended to be encompassed by the following claims.

1-101. (canceled)

102. A method for the preferential expansion of fetal cells over maternal cells, comprising preferentially expanding fetal cells over maternal cells by incubating cells from a sample of maternal blood containing CD34+ cells of both maternal and fetal origin in the presence of Stem Cell Factor (SCF) in a serum free media.

103. The method of claim 102, wherein the fetal cells are preferentially expanded by a factor of at least about 3 compared with maternal cells.

104. The method of claim 102, wherein the fetal cells are preferentially expanded by a factor of at least about 5 compared with maternal cells.

105. The method of claim 102, wherein the cells from a sample of maternal blood are incubated in the presence of SCF at a concentration of from about 15 ng/ml to about 250 ng/ml.

106. The method of claim 102, wherein the cells from a sample of maternal blood are incubated in the presence of SCF at a concentration of about 100 ng/ml.

107. The method of claim 102, further comprising incubating the cells from a sample of maternal blood in the presence of one or more of the following: IL-3, IL-6, EPO, TPO, FIt-I, Flt-3, IL-I, IL-11, GM-CSF, G-CSF, Wnt, Notch, IGF, BMP, Sonic Hedgehog, CxCL12, basic fibroblast growth factor, specific vitamins, and specific antibodies capable of inhibiting adult cell growth.

108. The method of claim 102, further comprising incubating the cells from a sample of maternal blood in the presence of at least one of EPO and TPO.

109. The method of claim 102, wherein the serum free media comprises Hematopoietic Progenitor Growth Medium (HPGM).

110. The method of claim 102, wherein the fetal cells are expanded in the absence of substantial expansion of the maternal cells.

111. The method of claim 102, further comprising enriching the fetal cells from a sample of maternal blood by at least one of the following: removing or lysing red blood cells; selecting or sorting cells based on the presence or absence one or more marker; or immunomagnetic selection.

112. The method of claim 111, wherein CD34+ cells are enriched from a sample of maternal blood by at least one of the following: positive selection of CD34+ cells, direct selection of CD34+ cells, indirect selection of CD34+ cells, depletion of non-CD34+ cells, or depletion of CD34− cells.

113. The method of claim 111, wherein the fetal cells are enriched from the sample of maternal blood by selecting or sorting cells based on the presence or absence of one or more of the following markers: CD34, CD133, CD117, CD2, CD90, CDIc, CD14, CD24, CD48, CD86, CD235a, MPO, MS4A6A, MS4A7, or ASGR2.

114. The method of claim 102, further comprising the steps of:
detecting the preferential expansion of fetal cells over maternal cells; and
analyzing one or more of the fetal cells for one or more characteristics.

115. The method of claim 114, wherein the one or more characteristics comprises a genotype, phenotype, physiological function, or biochemical function.

116. The method of claim 114, wherein the one or more characteristics comprises a disease or condition or an indicator of a disease or condition.

117. The method of claim 114, wherein the one or more characteristics comprises a chromosomal abnormality.

118. The method of claim 114, further comprising selecting or sorting the fetal cells from the maternal cells based on one or more of the following cell markers: CD34, CD133, CD117, CD2, CD90, CDIc, CD14, CD24, CD48, CD86, CD235a, MPO, MS4A6A, MS4A7, and ASGR2.

119. The method of claim 114, further comprising harvesting one or more colonies of fetal cells formed during the preferential expansion.

120. A composition comprising a mixture of fetal and maternal stem cells formed by incubating cells from a sample of maternal blood containing CD34+ cells of both maternal and fetal origin in the presence of SCF in a serum free media, wherein the fetal stem cells are preferentially expanded by a factor of at least about 3 compared with the maternal stem cells.

121. The composition of claim 120, wherein the fetal stem cells are preferentially expanded by a factor of at least about 5 compared with the maternal stem cells.
